(19) United States
(12) Patent Application Publication
(10) Pub. No.: US 2010/0205678 A1
(43) Pub. Date: Aug. 12, 2010

(54) OOCYTE SPINDLE-ASSOCIATED FACTORS IMPROVE SOMATIC CELL CLONING

(76) Inventors: Eric W. Overstrom, Grafton, MA (US); Elena Ibáñez, Terrassa (ES); Christine A. Lima, Pawtucket, RI (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/448,699

(22) PCT Filed: Jan. 4, 2008
(86) PCT No.: PCT/US2008/000119

§ 371 Date: Apr. 22, 2010

Related U.S. Application Data
(60) Provisional application No. 60/879,267, filed on Jan. 5, 2007.

(51) Int. Cl. C12N 15/06; A01K 67/027; C12P 21/00; C12N 5/07

(52) U.S. Cl. 800/4; 435/449; 800/24; 435/375

(57) ABSTRACT

The invention pertains to the discovery that the presence of oocyte spindle associated factors in an enucleated oocyte improves oocyte quality and subsequently nuclear transfer. In particular, it was discovered that maintaining oocyte spindle factors in the oocyte after enucleation improves oocyte quality for use in nuclear transfer methodology.

### RELATED APPLICATION

This application claims the benefit of U.S. Provisional Application No. 60/879,267, filed on Jan. 5, 2007. The entire teachings of the above application are incorporated herein by reference.

### BACKGROUND OF THE INVENTION

Nuclear transfer methods have been developed and used successfully to produce cloned animals, in particular, sheep, cattle, mice, goats and pigs. In these methods, typically a donor nuclear genome (karyoplast), and an enucleated oocyte (cytoplast) are combined to produce a cloned embryo.

Mammalian oocyte cytoplasts have been prepared by physically removing nuclear chromatin by micromanipulation techniques in preparation to receive the donor genome. Enucleated oocytes arrested at metaphase of Meiosis II (MID are subsequently “reconstructed” by the addition of the donor karyoplast typically using either electrofusion or microinjection techniques. However, physical enucleation is generally technically demanding, time consuming, inherently invasive and damaging to cytoplast spatial organization. Moreover, in certain instances, development of reconstructed embryos is inefficient.

In traditional somatic cell nuclear transfer (SCNT), oocytes arrested at metaphase of Meiosis II (MID are stained with Hoechst 33342 and exposed to UV irradiation to cause the fluoresce of the chromatin. Under constant UV exposure, the oocyte is punctured and its chromosomes are manually removed with a fine bore glass pipet creating an enucleated egg or cytoplast. The cytoplast is then injected with DNA or fused to a somatic cell. The reconstructed embryo is then stimulated to continue development into an embryo and beyond. This process is technically difficult, requiring expensive equipment and significant micromanipulation training. Not only is this technique a very labor intensive process, but it is widely believed that due to the invasiveness of this method, the egg may be irreversibly damaged beyond the point where healthy development can be sustained.

One alternative strategy to physical enucleation has been to treat oocytes with agents that modify the processes of karyokinesis and cytokinesis and result in chemically enucleated oocytes at high rates (>85%). However, certain studies have reported that exposure of Metaphase I and MII oocytes to etoposide, a Topoisomerase II inhibitor, and cycloheximide yields enucleated cytoplasts with limited ability to support cleavage or blastocyst development, and term development of reconstructed embryos has not been reported. However, this method can also result in limited success with producing viable offspring.

In spite of recent advances in cloning methodology, embryonic development remains delayed and variable. Abortions and placental abnormalities are common, increased birth weight is common (known as “large offspring syndrome”), and resultant newborns commonly exhibit defects. Nuclear transfer presents challenges due to the technical complexity, multiple-step protocols, inconsistent oocyte quality, equipment costs, health of the cloned animals and ethical and moral contradictions.

Hence, a need exists for improved methods for developing nuclear transfer embryos with increased efficiency. In particular, competent oocytes are needed that when combined with donor nucleic are able to produce viable offspring without the abnormalities and complications.

### SUMMARY OF THE INVENTION

The invention pertains to the discovery that the presence of oocyte spindle-associated factors in the enucleated oocyte improve nuclear transfer. In particular, it was discovered that oocyte spindle factors improve oocyte quality, or developmental competence, for use in nuclear transfer methodology.

The invention pertains to a method of forming a nuclear transfer embryo obtaining an enucleated oocyte, containing and maintaining an effective amount of spindle-associated enabling factors in the enucleated oocyte, and combining the enucleated oocyte and at least the nucleus of a donor cell of the same species of said oocyte, thereby forming a nuclear transfer embryo. The spindle-associated factors are selected from the group, many of which are cell cycle-regulated chromosomal passenger proteins, consisting of: Aurora kinase A, Aurora kinase B, Aurora kinase C, Survivin, Securin, INCEP, Borealin/Dasra B, Bora, gamma tubulin, pericentrin, members of the Rec8 family proteins, Cdc20, members of the Anaphase Promoting Complex (Apc), the Polo kinases, Feo/Klp3A, cohesin, MEI-S322, spindle cell-cycle checkpoint proteins, Bub1, Bub3, BubR1, Mad1, Mad2 and CENP-E and combinations thereof. In certain embodiments, the oocytes are enucleated with a chemical selected from the group consisting of demecolcine, paclitaxel, phalloidin, colchicine, and nocodozole. In other embodiments, the method includes activating the oocyte prior to exposing the oocyte to said chemical. In certain embodiments, the oocyte is mammalian, such as a non-human.

The invention also pertains to a method of cloning a mammal, comprising: obtaining an enucleated oocyte, maintaining an effective amount of spindle-associated factors in the enucleated oocyte, and combining the oocyte with at least the nucleus of a donor cell of the same species of said oocyte prior to cessation of extrusion of the second polar body from said oocyte, thereby forming a nuclear transfer embryo, impregnating a mammal of the same species as the nuclear transfer embryo with the nuclear transfer embryo under conditions suitable for gestation of the cloned mammal; and gestating the embryo, thereby causing the embryo to develop into the cloned mammal.

The invention also relates to a method of producing a transgenic mammal, comprising: destabilizing microtubules of an oocyte, whereby essentially all endogenous genetic material collects at a second polar body during meiosis of said oocyte and maintaining spindle-associated factors in resulting enucleated oocyte; and combining the oocyte with at least the nucleus of a donor cell of the same species of said oocyte prior to cessation of extrusion of the second polar body for said oocyte, thereby forming a nuclear transfer embryo, impregnating a mammal of the same species as the nuclear transfer embryo with the nuclear transfer embryo under conditions suitable for gestation of the transgenic mammal; and gestating the embryo, thereby causing the embryo to develop into the transgenic mammal.

In another embodiment, the invention includes a method of producing a protein of interest in an animal, comprising destabilizing microtubules of an oocyte, whereby essentially all endogenous genetic material collects at a second polar body during meiosis of said oocyte, maintaining spindle-associated factors in resulting enucleated oocyte; and combining the oocyte with at least the nucleus of a donor cell of the same species of said oocyte prior to cessation of extrusion of the second polar body from said oocyte, thereby forming a nuclear transfer embryo, impregnating a mammal of the same species as the nuclear transfer embryo with the nuclear transfer embryo under conditions suitable for gestation of the cloned mammal; gestating the embryo, thereby causing the embryo to develop into the cloned mammal; and purifying the protein of interest from the cloned animal.

### BRIEF DESCRIPTION OF THE DRAWINGS

As used herein, if an orange arrow is recited, a reference “A” has been incorporated in the appropriate figures. As used herein, if a yellow arrow is recited, a reference “B” has been incorporated in the appropriate figures.

FIG. 1 is a schematic drawing of the initiation of Anaphase by the Apc.

FIG. 2 is a schematic drawing of Apc activity.

FIG. 3 is a series of slides illustrating negative control images of HeLa cells. Unsynchronized HeLa cells were imaged without secondary or primary antibodies in the staining protocol: Apc11 alone (A), Cdc20 alone (B), Rec8 alone (C), or Alexa594 Goat anti-Rabbit IgG alone (D). DIC images (left) are shown with 5 ms exposure; Red fluorescence images (right) are shown with a 100 ms exposure. Single cells are circled with a white dash. The background has been set to black.

FIG. 4 is a series of slides illustrating localization of Apc subunits in unsynchronized HeLa cells. Apc11 (A) and Cdc20 (B) are shown in red. Hoechst 22358 (Chromatin) is shown in blue. Evidence of colocalization is purple. G₀ cells show a cytoplasmic distribution of Apc subunits while cells undergoing cellular replication show evidence of colocalization around the chromatin. Orange arrows point to the kinetochores in prophase cells. Yellow arrows point to the spindle in a cell in metaphase. Red fluorescence is imaged with a 100 ms exposure.

FIG. 5 is a series of slides illustrating Apc3 localization. Apc3 (left panel, green) was localized in synchronized HeLa cells. CREST staining of centromeres is shown in the middle panels. The right panel is the merge of the two. At prophase (A), Apc3 staining is detected at the kinetochores while at metaphase (B), staining is detected at the mitotic spindle.

FIG. 6 is a slide illustrating localization of Rec8 in unsynchronized HeLa cells. Anti-Rec8 is stained in red. Hoechst 22358 (Chromatin) is stained in blue. At metaphase, Rec8 (red) is highly expressed around the chromosomes (blue) aligned around the metaphase plate (yellow arrow). In non-dividing cells, Rec8 staining is less distinct in the cytoplasm. Red fluorescence is imaged with a 100 ms exposure.

FIG. 7 is a series of slides illustrating negative control images of oocytes. Oocytes were imaged without the addition of secondary and/or primary antibodies in the staining protocol: neither primary nor secondary antibody (A), Apc11 alone (B), Cdc20 alone (C), or Rec8 alone (D) without the addition of secondary antibody. Similar images were obtained from oocytes stained with only secondary antibodies (data not shown). DIC images (left) are shown with 5 ms exposure; fluorescence images (right) are shown with a 150 ms exposure. Single cells are circled with a white dash. The background has been set to black.

FIG. 8 is a series of slides illustrating Apc11 staining optimization in mouse oocytes. Oocytes fixed in 2% PFA with Triton X-100 were incubated in decreasing:concentrations (1:1000, top panel), (1:2000, middle panel), (1:4000, bottom panel) of Apc11, either overnight at 4° C. or for 1 hour at either room temperature or 37° C. Oocytes were stained for Apc11 (green), Hoechst 22358 (blue), and tubulin (red). Oocyte autofluorescence appears as a dull green haze. Scale bars are 15 μm. All green fluorescence were imaged with a 100 ms exposure.

FIG. 9 is a pair of slides illustrating oocytes fixed with MTSB-XF. Oocytes were fixed at MII with MTSB-XF and stained with anti-Apc11 (green), Hoechst 22358 (blue), and Phalloidin labeled with Texas Red (red). While Apc11 localization can still be detected in an exclusion zone around the meiotic spindle, the overall staining pattern is non-distinctive.

FIG. 10 is a pair of slides illustrating localization of Apc11 in oocytes arrested at metaphase of Meiosis II (MID. Denuded oocytes were fixed in PFA immediately following removal from hyaluronidase and stained for Apc11 (green), chromatin (blue), and α/β tubulin (red). The left panel shows Apc11 alone; the right panel is the overlay of the three stains. Yellow arrows indicate perispindular localization. The orange arrow indicates the hemispheric ridge. Green fluorescence is imaged at 120 ms.

FIG. 11 is a pair of slides illustrating localization of Apc11 in oocytes fixed at Anaphase of Meiosis II (AII). Denuded oocytes were fixed in PFA 25 minutes after the initiation of activation. Oocytes were stained for Apc11 (green), chromatin (blue), and α/β tubulin (red). The left panel shows Apc11 alone; the right panel is the overlay of the three stains. Green fluorescence is imaged at 120 ms.

FIG. 12 is a pair of slides illustrating localization of Apc11 in oocytes fixed at telophase of Meiosis II (TII). Denuded oocytes were fixed in PFA 2 hours after the initiation of activation. Oocytes were stained for Apc11 (green), chromatin (blue), and α/β tubulin (red). The left panel shows Apc11 alone; the right panel is the overlay of the three stains. Green fluorescence is imaged at 120 ms.

FIG. 13 is a pair of slides illustrating localization of Apc11 in oocytes fixed in Interphase. Denuded oocytes were fixed in PFA 4 hours after the initiation of activation. Oocytes were stained for Apc11 (green), chromatin (blue), and α/β tubulin (red). The left panel shows Apc11 alone; the right panel is the overlay of the three stains. Green fluorescence is imaged at 120 ms.

FIG. 14 is a pair of slides illustrating effects of demecolcine on Apc11 localization in oocytes fixed at Anaphase II (AII). Denuded oocytes were incubated in 10 mM SrCl₂ for 10 minutes followed by a 15-minute incubation in media containing 10 mM SrCl₂ and 0.4 μg/mL demecolcine (left panel). Apc11 alone (right panel). Overlay of Apc11 (green), Hoechst 22358 (blue), and α/β tubulin (red). Green fluorescence is imaged at 120 ms.

FIG. 15 is a pair of slides illustrating effects of demecolcine on Apc11 localization in oocytes fixed at TII. Denuded oocytes were incubated in 10 mM SrCl₂ for 10 minutes, followed by a 110-minute incubation in media containing 10 mM SrCl₂ and 0.4 mg/mL demecolcine (left panel). Apc11 alone (right panel). Overlay of Apc11 (green), Hoechst 22358 (blue), and α/β tubulin (red). Green fluorescence is imaged at 120 ms.

FIG. 16 is a pair of slides illustrating effects of demecolcine on Apc11 localization in oocytes fixed at Interphase. Denuded oocytes were incubated in 10 mM SrCl₂ for 10 minutes followed by a 230-minute incubation in media containing 10 mM SrCl₂ and 0.4 μg/mL demecolcine. (left panel) Apc11 alone. (right panel). Overlay of Apc11 (green), Hoechst 22358 (blue), and α/β tubulin (red). Green fluorescence is imaged at 120 ms.

FIG. 17 is a pair of slides illustrating localization of Cdc20 in oocytes fixed at MII. Denuded oocytes were fixed immediately following removal from hyaluronidase. (left panel) Cdc20 alone. (right panel) Overlay of Cdc20 (red), Hoechst 22358 (blue), and α/β tubulin (green). At MII, Cdc20 stained shows punctate spots throughout the cytoplasm. Red fluorescence is imaged at 100 ms.

FIG. 18 is a pair of slides illustrating localization of Cdc20 in oocytes fixed at Anaphase II (AII). Denuded oocytes were fixed after a 25-minute incubation in 10 mM SrCl₂. (left panel) Cdc20 alone. (right panel) Overlay of Cdc20 (red), Hoechst 22358 (blue), and α/β tubulin (green). At AII, Cdc20 staining shows diffuse cytoplasmic localization. Red fluorescence is imaged at 150 ms.

FIG. 19 is a pair of slides illustrating localization of Cdc20 in Oocytes fixed at Telophase II (TII). Denuded oocytes were fixed after a 1-hour incubation in 10 mM SrCl₂. (left panel) Cdc20 alone. (right panel) Overlay of Cdc20 (red), Hoechst 22358 (blue), and α/β tubulin (green). At TII, Cdc20 staining shows a dim cytoplasmic haze not significantly brighter than background autofluoresce (data not shown). Red fluorescence is imaged at 250 ms.

FIG. 20 is a pair of slides illustrating localization of Cdc20 in oocytes fixed at Interphase. Denuded oocytes were fixed after a 4-hour incubation in 10 mM SrCl₂ (left panel). Cdc20 alone (right panel). Overlay of Cdc20 (red), Hoechst 22358 (blue), and α/β tubulin (green). Red fluorescence is imaged at 150 ms.

FIG. 21 is a pair of slides illustrating effects of demecolcine on Cdc20 localization in oocytes fixed at Anaphase II (AII). Denuded oocytes were incubated in 10 mM SrCl₂ for 10 minutes followed by a 15-minute incubation in media containing 10 mM SrCl₂ and 0.4 μg/mL demecolcine (left panel). Cdc20 alone (right panel). Overlay of Cdc20 (red), Hoechst 22358 (blue), and α/β tubulin (green). Red fluorescence is imaged at 100 ms.

FIG. 22 is a pair of slides illustrating effects of demecolcine on Cdc20 localization in oocytes fixed at Telophase II (TII). Denuded oocytes were incubated in 10 mM SrCl₂ for 10 minutes followed by a 110-minute incubation in media containing 10 mM SrCl₂ and 0.4 μg/mL demecolcine (left panel). Cdc20 alone (right panel). Overlay of Cdc20 (red), Hoechst 22358 (blue), and a/P tubulin (green). Red fluorescence is imaged at 100 ms.

FIG. 23 is a pair of slides illustrating effects of demecolcine on Cdc20 localization in oocytes fixed at Interphase. Denuded oocytes were incubated in 10 mM SrCl₂ for 10 minutes followed by a 230-minute incubation in media containing 10 mM SrCl₂ and 0.4 μg/mL demecolcine (left panel). Cdc20 alone (right panel). Overlay of Cdc20 (red), Hoechst 22358 (blue), and α/β tubulin (green). Red fluorescence is imaged at 100 ms.

FIG. 24 is a pair of slides illustrating spatial localization of Rec8 in oocytes arrested at MII. Denuded oocytes were fixed immediately following removal from hyaluronidase. Overlays of Rec8 (red) and Hoechst 22358 (blue). At MII, Rec8 localizes to the cortical region directly overlying the condensed chromatin in both the meiotic spindle (yellow arrows) and first polar body (orange arrow). Red fluorescence is imaged at 120 ms.

FIG. 25 is a series of slides illustrating spatial localization of Rec8 in oocytes fixed at Anaphase II (AII). Denuded oocytes were fixed after a 10-minute (top panel), a 20-minute (middle panel), and a 40-minute (bottom panel) incubation in 10 mM SrCl₂. Overlays of Rec8 (red) and Hoechst 22358 (blue). During Anaphase II, Rec8 localizes to an area surrounding chromatin that will become the female pronucleus (yellow arrows) and the chromatin that is to be extruded in the second polar body (orange arrows). The first polar body is highlighted with an aqua dashed circle. Red fluorescence is imaged at 120 ms.

FIG. 26 is a slide illustrating spatial localization of Rec8 in oocytes fixed at Telophase II (TII). Denuded oocytes were fix in PFA after a 2-hour incubation in 10 mM SrCl₂. Rec8 is shown in red. Chromatin (Hoechst 22358) is shown in blue. In TII eggs, Rec8 localized around the female pronucleus and the budding second polar body similar to the late Anaphase oocyte in FIG. 25. Red fluorescence is imaged at 120 ms.

FIG. 27 is a pair of slides illustrating spatial localization of Rec8 in oocytes fixed at Interphase. Denuded oocytes were fixed after a 4-hour incubation in 10 mM SrCl₂. Rec8 alone (left panel). Overlay of Rec8 (red) and Hoechst 22358 (blue) (right panel). During Interphase, some Rec8 remains localized to the female pronucleus (yellow arrow), whereas some disperses throughout the cytoplasm. Red fluorescence is imaged at 200 ms.

FIG. 28 is a pair of slides illustrating the effect of demecolcine on Rec8 localization in oocytes fixed at Anaphase II (AII). Oocytes were incubated for 10 minutes in 10 mM SrCl₂ followed by a 10-minute incubation in media containing both SrCl₂ and 0.4 μg/mL demecolcine before being fixed at AII. Rec8 alone (left panel). Overlay of Rec8 (red) and Hoechst 22358 (blue) (right panel). Note the localization of Rec8 near the chromatin, but not directly on it. Red fluorescence is imaged at 120 ms.

FIG. 29 is a pair of slides illustrating the effect of demecolcine on Rec8 localization in Telophase II (TII) eggs. Oocytes were incubated for 10 minutes in 10 mM SrCl₂ followed by a 70-minute incubation in media containing both SrCl₂ and 0.4 μg/mL demecolcine before being fixed at TII. Overlays of Rec8 (red) and Hoechst 22358 (blue). In TII eggs treated with demecolcine, Rec8 colocalizes (yellow arrows) directly with chromatin (not the surrounding area). Red fluorescence is imaged at 150 ms.

FIG. 30 is a pair of slides illustrating the effect of demecolcine on Rec8 localization in oocytes fixed in Interphase. Oocytes were incubated for 10 minutes in 10 mM SrCl₂ followed by a 230-minute incubation in media containing both SrCl₂ and 0.4 μg/mL demecolcine. Rec8 alone (left panel). Overlay of Rec8 (red) and Hoechst 22358 (blue) (right panel). Rec8 appears to show some localization directly on the chromatin (similar to FIG. 29). Red fluorescence is imaged at 150 ms. The foregoing will be apparent from the following more particular description of example embodiments of the invention, as illustrated in the accompanying drawings, in which like reference characters refer to the same parts throughout different views. The drawings are not necessarily to scale, instead emphasis is being placed upon illustrating embodiments of the present invention.

FIG. 31 is a schematic demonstrating a comparison of nuclear transfer methods.

FIG. 32 is a schematic showing convention enucleation.

FIG. 33 is a schematic demonstrating induced enucleation.

FIG. 34 is a slide demonstrating oocyte quality, showing the kinetics of spindle rotation.

FIG. 35 is a schematic showing localization of Aurora A and B.

FIG. 36 is a slide showing immunofluorescence controls.

FIG. 37 is a slide showing Rec8 expression in oocytes.

FIG. 38 is a slide showing Apc11 expression in oocytes.

FIG. 39 is a slide showing Cdc20 expression in oocytes.

FIG. 40 is a schematic demonstrating cytoplasmic centrosomes from different stains.

FIG. 41 is a slide showing spindle characteristics in IVO, IVM and IVM+.

FIG. 42 is a slide depicting the Hesperadin treatment on oocytes.

FIG. 43 is a slide showing samples fixed following 6 h treatment and maturation.

FIG. 44 is a slide showing samples fixed following 10 h treatment and maturation.

FIGS. 45-46 graphically depict mRNA levels of Aur A, B, and C in MII oocytes, activated oocytes and cumulus cells.

FIG. 47 graphically depicts mRNA levels of Aur A, B and C in germinal vesicles (GV) with and without the zona pellucida (ZP).

### DETAILED DESCRIPTION OF THE INVENTION

A description of example embodiments of the invention follows.

The present invention relates to methods of maintaining oocyte spindle-associated factors in the enucleated oocyte for subsequent reconstruction with donor nuclei. Oocyte induced enucleation maintain spindle-associated regulators or factors that provide for cytoplast developmental competence. These enabling factors have a role in improved methodology in nuclear transfer.

Traditional enucleation methods removed the oocyte chromatin and surrounding cytoplasm containing spindle-associated factors and consequently displayed poor competence and poor efficiency. For example, exposure to UV light used in traditional methods has been shown to negatively affect oocyte competence in several species by disturbing membrane processes, intracellular elements, and mitochondrial chromatin (Smith, J Reprod Fertil. 99(1): 39-44. (2003); Velilla et al., Zygote, 10(3): 201-208.2002). Additionally, the manual removal of the MII chromosomes is imprecise. During this process, the meiotic spindle, the surrounding cytoplasm, and any other cellular components associated with the meiotic spindle are also removed from the egg.

Although traditional methods have been successfully employed to produce a variety of organisms including sheep (Wilmut et al., Nature, 385: 810-813 (1997)), cows (Kato et al., Science, 282: 2095-2098 (1998)), goats (Baguisi et al, 1999; Lan et al., Mol Reprod Dev., 73(7): 834-840 (2006) and several others), the results have shown poor efficiency. For example, the efficiency of sheep was: 0.4-2%, cattle: 1-6%, mice: 2-5%, goats: 2.6-5.7%, and pigs: 0.6-4.8%. Thus, nearly a decade after Wilmut et al. (1997) reported the birth of Dolly the sheep, the first live mammal cloned from an adult cell, the efficiency of this technique remains exceedingly low (2-5%), despite the variety of cloning methods employed (reviewed by Kato et al., 1999; Campbell et al., Reprod. Dom. Anim., 40: 256-268 (2005)).

The use of demecolcine, a derivative of colchicine, aids in the enucleation procedure and demonstrates an increase in the efficiency of SCNT. See U.S. Published Patent Application No. US 2004/0019924, Russell et al., Mol Reprod Dev.; 72(2):161-70 (2005), Ibanez et al., Reproduction, 2005 December; 130(6):845-55 and Ibanez et al., Biol Reprod. 2003 April; 68(4):1249-58. In this method, the MII oocytes were incubated in demecolcine to depolymerize the meiotic spindle and the oocytes were subsequently activated. In other methods, Teleophase II oocytes are used. It was observed that oocytes extruded the chromatin in the second polar body with a high efficiency. Using this method, it was suggested that the karyoplast could then be removed more easily than in traditional physical manipulation methods. Demecolcine-assisted enucleation has since been effective in several species including mice (Baguisi & Overstrom, 2000), sheep (Hou et al., Reprod, Nutr, Dev. 46(2): 219-26 (2006)), and cows (Russell et al., Molecular Reproduction and Development, 72: 161-170. 2005). Other methods of destabilizing microtubules can also be utilized for obtain an enucleated oocyte. For example, the microtubules can be destabilized by exposing the microtubules to electromagnetic radiation, x-rays and/or heat. Further, the microtubules can be destabilized by exposure of the oocyte to a change in pH or osmolality.

Oocyte-enabling competencies are important in nuclear transfer. Nuclear reprogramming is needed for gene expression and epigenetics. Competent proliferation is needed for cell cycle progression and checkpoint control and fidelity. Unfortunately, using traditional techniques, many of these cytoplasmic components, that assist with the developmental competence of the enucleated oocyte, are removed. The absence of spindle factor contributes to the cytoplast's inability to support later development. As described herein, maintaining the presence of native spindle-associated factors shows improved cell cloning. In certain embodiments, induced enucleated oocytes with spindle-associated factors are combined with donor nuclei to form a nuclear transfer embryo.

The present invention shows that the presence of spindle-associated factors in the oocyte further improves somatic cell cloning. The actions of the factors can be studied and localized using known methods in the field, for example: utilizing knockout studies using RNAi, using Western blotting with antibodies directed to the factors, studying the action of inhibitors, utilizing traditional staining techniques to observe localization of the factors during various stages of oocyte development and nuclear transfer, as well as other methods available. These methodologies disable, remove or inhibit the factors for studying the particular role of spindle factors in oocyte competence.

### Spindle-Associated Factors

A number of factors are known to be associated with the spindle. The following is a list of certain factors, although the invention is not limited by the following list: Aurora kinases (A, B and C), Survivin, Securin, INCEP, Borealin/DasraB, Bora, gamma tubulin, pericentrin, Rec8 family proteins, Cdc20, Apc complex, including Apc11, cohesin, MEI-S322, and the spindle checkpoint proteins, Bub1, Bub3, BubR1, Mad1, Mad2 and CENP-E.

To better understand the effects of demecolcine (and other MT-destabilizing drugs) on the spindle-associated factors, various experiments to measure the activity and inter-relationship of these factors are needed. For example, western blots should be designed to measure relative protein concentrations of the factors and related family members at all stages of development. These concentration changes would be closely monitored in the presence and absence of microtubule-destabilizing chemicals, such as demecolcine to determine if the drug interferes with the orderly destruction of these and other proteins in the oocyte during development.

Understanding the temporal role of each of these factors will lead to increased efficiency of cloning methodologies.

Additionally, RT-PCR and RNAi experiments could be developed to study gene expression of the Anaphase the key cell cycle regulators in the oocyte.

### The Aurora Kinases

The Aurora family of kinases comprise three serine/threonine kinases, each with roles in cell cycle progression and completion, in particular they are involved in controlling M phase progression. Aurora A (AurA), localized to the spindle poles, is responsible for proper chromosome segregation. Aurora B (AurB), a member of the chromosomal passenger complex, is required for Metaphase plate alignment and cytokinesis. Less is known about Aurora C (AurC), but it is believed to have actions similar to Aurora B. Aurora A is localized in Prophase and Prometaphase, while Aurora B is localized in Metaphase, Anaphase and Cytokinesis. Previous reports have noted aberrant spindle morphology associated with Aurora A inhibition, while Aurora B mutations have been shown to yield atypical chromosome segregation with eventual failure of Cytokinesis in somatic cells. It is well established that phosphorylation of histone H3 on ser10 (pH3) is a marker of Aurora B activity.

In mouse and xenopus oocytes, Aurora A is well characterized and localization is identical to that seen in mitotic cells. Loss of Aurora A by antibody microinjection resulted in variable disorganized spindles. Microtubule destabilization resulted in disorganized spindles and Aurora A diffusion throughout the cytoplasm (Yao et al., Biol. Repro, 2004).

Aurora B was studied in C. elegans. oocytes, and the localization was identical to that seen in mitotic cells but only in the presence of sperm in the spermatheca. Otherwise, diffuse cytoplasmic staining was observed. Loss of Aurora B by RNAi resulted in a frequent failure of Cytokinesis completion.

The actions of Aurora kinases can be studied using known methods, for example the use of the broad spectrum inhibitor Hersperadin. Hersperadin is known to block Aurora A and Aurora B and most likely affects Aurora C. (Lima et al., Society for the Study of Reproduction's journal, Biology of Reproduction 2006 Special Issue for the annual meeting July 29-August 1).

Previous studies have demonstrated particular phenotypes in treated mammalian cells. For example, abnormal spindles, improperly attached chromosomes and aneuploidy have been seen (Rosa et al, MBC, 2006).

Example 2 describes the action of Hesperadin on mouse oocytes.

### Survivin

Survivin is a member of the chromosomal passenger complex (CPC). In mammalian cell culture, expression is cell cycle regulated with a peak during M phase. In many cancer cells Survivin is up-regulated. Previous studies have knocked out Survivin. In these studies, embryonic lethality in mice was seen. In HeLa cells, spindle organization, chromosome alignment, and Cytokinesis defects were observed (Klein et al., Mol. Biol. Cell., EPub. (2006)). Survivin is also a member of the inhibitor of apoptosis (IAP) family of proteins. Thus, there is conflicting data on Survivin's actual role.

### Anaphase-Promoting Complex (Apc)

### Apc in Somatic Cells

The Anaphase-promoting complex (Apc) is a multi-subunit protein that is crucial in the regulation of the cell cycle (Peters, 2002), with subunit Apc11 serving as the catalytic core (reviewed by Castro et al., 2005). The Apc is an E3 ubiquitin ligase that marks target proteins for degradation by the 26S proteasome. The irreversibility of proteolysis is utilized by cells to give the cell cycle directionality. In somatic cells, the main function of the Apc is the ubiquitination of cyclins (specifically cyclin B) and Securin. Ubiquitin is a 76aa molecule that acts as a signal that causes the target protein to be transported to a proteasome for degradation (Chau et al., 1989). The destruction of cyclin B leads to the inactivation of Cdk1, a cyclin-dependent kinase that initiates M phase in eukaryotic cells (Zachariae & Nasmyth, 1999). The inactivation of Cdk1 during Anaphase and Telophase is necessary for both the formation of prereplicative complexes and chromosome decondensation (Peters, 2002). Hence, the Apc indirectly leads to the inactivation of Cdk1 by marking cyclin B for destruction.

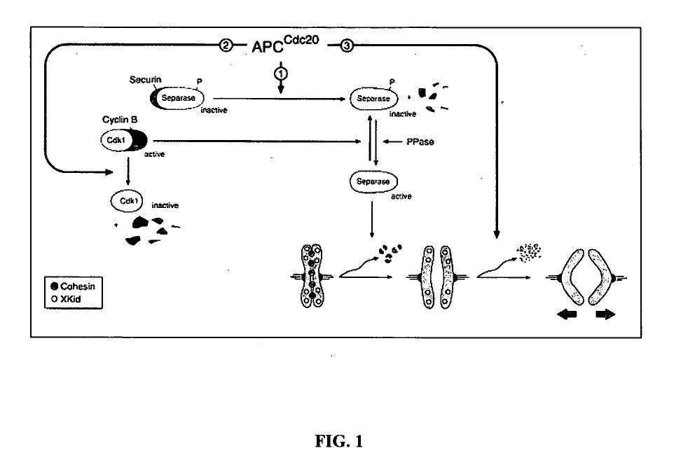

The other main function of the Apc in somatic cells is to label and destroy Securin. Since Securin binds and inhibits separase, its destruction indirectly activates the protease. Separase works to cleave SCC1 (Rec8 in meiotic cells), a subunit of the cohesion processes that hold sister chromatids together from Metaphase until Anaphase (Peters, 2002). Additionally, since Cdk1 initiatorily phosphorylates separase, the Apc affects separase activity in two ways: by the reduction of cyclin B concentrations and the destruction of Securin. (See FIG. 1).

There are two mitotic specificity factors for the Apc, which target different sets of proteins and are regulated differently: Cdc20 and Cdh1.

Cdc20 binds to Apc early in Mitosis to activate it. It is not clear what kinases phosphorylate and activate the Cdc20-Apc complex. It is known that M-Cdk is required for the activity of these kinases, although there is a significant delay between M-CdK activation and the activation of the Cdc20-Apc complex. The molecular basis of the delay is unknown, but is believed to involve the key to the correct timing of Anaphase initiation.

The Apc is activated at different parts of the cell cycle by the binding of Cdc20 and Cdh1. Early in Mitosis when Cdk activity is high, the Apc binds Cdc20 and actively binds proteins with a destruction box (D-box), the aa sequence R-x-x-L-x-x-x-x-N/D/E common to all the substrates of Apc^{Cdc20} (Harper et al., 2002). Apc^{cdc20} degrades A-type cyclins during prometaphase and B type cyclins and Securins during the beginning of metaphase (Peters, 2002). Alternatively, since Cdh1 is inhibited by Cdk activity, the Apc binds Cdh1 during G1, where Cdk activity is low. Similar to Apc^{Cdc20}, Aoc^{cdh1} also binds proteins with a specific sequence. That sequence, known as a KEN box (K-E-N-x-x-x-D/N) is common to all substrates of Apc^{cdh1} including Cdc20 (Peters, Molecular Cell, 9: 93-943 (2002)). Accordingly, since Apc^{cdh1} is responsible for the destruction of Cdc20, it helps to regulate the activity timing of Apc^{cdc20} (Harper et al., 2002). Since ApcCdc20 and Apc^{Cdh1} have different substrates, the Apc has the ability to remain active throughout the changing conditions of the cell cycle.

### Apc in M-II Eggs

In normal vertebrate egg development, an egg will proceed through all of the steps of Meiosis until it reaches a final step in which the cell can no longer advance without an external stimulus. This pause in development is known as the Metaphase II (M-II) arrest. This arrest is partially caused by cytostatic factor (CSF) which inhibits the Apc from degrading cyclin B. By maintaining high cyclin B-Cdc2 levels, the cells will remain at this arrest until fertilization. Upon fertilization, a series of Ca²⁺ signals initiate a cascade that ends in the destruction of cyclin B and the next cellular division (Nixon et al., 2002). Experiments with cyclin B mutants without the D-box domain have shown that, if cyclin B is not degraded, no pronuclei will form and the cell will not exit Meiosis after fertilization (Magdwick et al., 2004).

Hysop et al. (2004) propose a model for mammalian eggs in which the Ca²⁺ signal affects the activity of the Apc during a Metaphase arrest and not the 26S proteasome as earlier characterized in lower organisms (Chiba et al., 1999). Hysop et al. propose that the Ca²⁺ signal stimulates the loss of an Apc inhibitor. One potential inhibitor Hysop et al. mentioned was Emil because of a potential phosphorylation site by CaMKII, the known Ca²⁺ transducer at fertilization (Markoulaki, Dev Biol, 258(2): 464-74. (2003)). However, Ohsumi et al. has reported that the M-phase arrest stimulated by Emil is separate from a CSF arrest in frogs (Xenopus). (Ohsumi et al., Proc Natl Acad Sci USA. 101(34): 12531-12536. (2004).) If Emil is not the Apc inhibitor, it is also possible that CSF may be a novel Ca²⁺-dependent inhibitor of the Apc (Hysop et al., 2004). During Metaphase, Securin maintains the inactivity of separase, an Anaphase-specific protease, until all the chromosomes are properly aligned or the initiation of Anaphase (Wirth et al., Journal of Cellular Biology, 172(6): 847-60 (2006)). At the onset of Anaphase, the destruction of Securin (regulated by Apc ubiquitination) allows separase to cleave the SCC1 subunit (Rec8 in meiotic cells) of cohesion, thus allowing sister chromatids to separate.

### Apc Subunits

### Apc11/Apc2 as the Catalytic Core

The cullin-RING domains (Apc2 and Apc11, respectively) of the Apc are believed to be the catalytic core of the complex (Gmachl et al., PNAS 97(16): 8973-8978 (2000); Leverson et al., 2000; Tang et al., Mol Biol Cell, 12: 3839-51 (2001)). Although Apc11 is among the smallest of the Apc subunits discovered (Passmore et al., 2005) Gmachl et al., 2000 have shown that recombinant human Apc11, and only Apc11, (not any of the other known subunits of the Apc) is sufficient for the synthesis of multiubiquitin chains in vitro in the presence of an E1 enzyme, Ubc4 and an ATP regenerating system. This synthesis occurred in both the presence and absence of substrates. However, these chains were non-specific as a D-box mutant of Securin was ubiquitinated as well as the wild type Securin. Additionally, Tang et al., 2001 coinfected Hi5 insect cells with viruses containing 10 Apc subunits. Combined, the multiple baculoviruses conveyed ubiquitin ligase activity. This activity was lost if only Apc2 or Apc11 were removed. Furthermore, Tang et al., 2001 showed that the Apc2/11 complex is sufficient for the ubiquitination of Securin with UbH10 as the E2 enzyme. Tang et al., 2001 then showed that while Ubc4 can interact directly with the RING of Apc11, UbH10 binds Apc2 strongly and weakly to Apc11.

Structure of Apc11 RING Finger

The E3 ubiquitin ligase activity of the Apc is conveyed by two Zn²⁺ ions binding within the RING domain of Apc11 and perhaps partially a third Zn²⁺ outside of the RING motif (Tang et al., 2001). When coordinating with these Zn²⁺ ions, a stable tertiary RING structure is formed. This RING structure is necessary for the ubiquitination of Apc substrates, as mutants with disrupted ring structures show significantly reduced to no ubiquitin ligase capability (reviewed by Peters, 2002). Although Tang et al., 2001 demonstrated that high levels of Zn²⁺ alone can catalyze minimal levels of a ubiquitination reaction in the presence of an E2, it is not yet known whether the RING structure of the Apc directly catalyzes the ligase reaction through the Zn ions or whether it allows for a stable proximity reaction to occur (Passmore & Barford, 2004).

### Structure of Apc2

As the second largest protein of the Apc (Jorgensen et al., Molecular and Cellular Biology: 468-476 (2001)), Apc2 is a protein with a cullin C-terminal homology region that binds strongly to Apc11 (Tang et al., 2001). All cullin proteins form a rigid scaffolding-like structure by binding the RING with their C-terminal domain, while the N-terminal region is thought to actively recruit the E2 enzymes (reviewed by Petroski & Deshaies, 2005). The structure of Apc2 has been inferred from its homology to Cull, another cullin protein in the SCF E3 ligase (Zheng et al., 2002). This inference is further supported by the fact that, while the sequence homology of the two proteins is mainly restricted to the C-terminal cullin domain (Passmore, 2004), a crystal structure of the C-terminal 78aa (well outside of the cullin region) forms a hinged-helix that can be superimposed over the same Cull region (Zheng et al., 2002). Along the C-terminus, Cull forms a V-shaped groove that binds Rbx1, a RING finger protein comparable to Apc11 (Zheng et al., 2002).

Along its N-terminus, Cull contains several helical repeats that are arranged to allow for the binding of Skp1, a linker protein that binds substrates of the SCF containing an F-box.

### Apc10 (Doc))

Apc10 is required for E3 ligase activity on certain substrates and plays a specific role in substrate recognition (Passmore et al., 2003). Apc10 interacts directly with Apc11, the catalytic core of the APC (Tang et al., 2001). Mutants of both fission and budding yeast lacking Apc10 show an arrest at Metaphase and the accumulation of mitotic cyclins (Kominami et al., 1998). Apc10 is the first member described in the Doc homology family, a group of proteins that have been detected in other E3 ligases unrelated to the Apc (reviewed by Passmore, 2004). Although its specific role is still undefined, Passmore et al., 2003 proposed that, since Apc10 mutants have a diminished ability to bind substrates, it functions as a regulator of substrate recognition. An additional report by Carroll & Morgon, 2002 shows that Apc10 increases processivity, and the addition of multiple ubiquitin molecules in a single binding event, by reducing substrate disassociation.

### Apc1 (Tsg24)

Apc1 is the largest subunit of the Apc (reviewed by Castro et al., 2005) and transiently localizes to the centromeres of mammalian chromosomes (Jorgenson et al., 1998) during Mitosis in CHO cells and throughout the cell cycle in murine cells. Its homologues include BimE from Aspergillus nidulans and Cut4 from Schizosaccharomyces pombe (reviewed by Castro et al., 2005). The predicted 3D structure contains Rpn1 and Rpn2, repetitive motifs that form a horseshoe-like structure (Jorgensen et al., 2001). While the exact function of this repetitive sequence is unknown, it has been predicted that this horseshoe might play a role in binding unfolded proteins, or as a scaffold for the rest of the Apc (Lupas et al., Trends in Biochemical Sciences, 22(6): 195-196 (1997)).

### Tetratricopeptide TPR Repeats (Apc3, Apc6, Apc7, Apc8)

The TPR sequence motif is found in proteins with various biochemical activities, and is thought to mediate protein-protein interactions (Castro et al., Oncogene 24: 314-325 (2005)). TPR sequences arrange themselves into anti-parallel α-helices that combine to form a right handed super helix (Das et al., EMBO Journal, 17(5): 1192-1199 (1988)). With specific aa residues on the outside and an extended grove inside the superhelix, the structure of multiple TPR sequences allows for the assembly of mult-protein complexes and the binding of an α-helix in the center. Specifically, Vodermaier et al., Current Biology, 13: 1459-1468 (2003) showed that Apc3 and Apc7 bind to the c-terminal isoleucine-arginine (IR) region of both Cdc20 and Cdh1, which are key activators of the Apc. Since all of these TPR subunits are phosphorylated during Mitosis, and that phosphorylation is necessary for the activation of the Apc, it is presumed that this phosphorylation event increases the binding ability of the Apc to Cdc20 (Kraft et al., 2003; reviewed by Castro et al., 2005). Interestingly, Apc10 also contains an IR tail signifying that Apc10 association is also mediated by the TPR subunits. Apc7 has only been described in vertebrates.

### Apc4, Apc5

Less is known about these subunits. It is hypothesized that these subunits, along with Apc1, connect Apc2 and Apc11 to the TPR subunits (Vodermaier et al., 2003).

### Apc9, Cdc26

Little is known about these two subunits other than the fact that they are required for overall structure of the Apc. Apc3 concentration is reduced in Apc9 and Cdc26 mutants, while Apc6 and Apc9 are reduced in Cdc26 mutants. So far, Apc9 has only been described in yeast.

### Apc13 (Swm1), Apc14, Apc14 (Mnd2)

Apc13, Apc14 and Apc15 are subunits that have only been described in yeast. While the biochemical function of these subunits is still unclear, it is hypothesized that they help maintain the structure of the Apc. Because the genes for Apc13 and Apc15 were originally identified in meiotic screens (Ufano et al., 1999; Rabitsch et al., 2001), a role for Apc13 and Apc15 in Meiosis has been predicted.

### Cdc20 (Fizzy)

Cdc20 binds to the Apc during Mitosis. Once bound, the Apc becomes activated to ubiquitinate substrates containing a D-box, which is a short aa sequence that promotes Apc recognition. The degradation of these substrates, including Securin, Xkid, and several cyclins, drives the cell through the mitotic cycle.

### Apc Localization and Activity

Previously, mitotic Apc localization has been observed in vitro (Tugendreich et al., 1995; Kraft et al., 2003; Acquiviva et al., 2004). The staining of Apc6 and Apc3 appears primarily on the centrosome at all cell cycle stages and coupled with the spindle following nuclear envelope breakdown (Tugendreich et al., Cell, 81: 261-268 (1995)). During Interphase, Apc3 staining is localized mainly to the nucleus and bound to the kinetochores in prophase. At pro-Metaphase, the staining appeared on the spindle (poles and fibers) and on the centromeres of chromatids that had not yet aligned on the Metaphase plate (Acquiviva et al., 2004). Acquiviva et al. (2004) went on to show that Apc3 localization could be eliminated in mutant cells without an active spindle checkpoint.

It is widely believed that Apc3 localization is necessary for the function of the Apc (reviewed by Pines & Lindon, Nat. Cell Biol., 7: 731-735 (2005)). One proposed mechanism of the RING E3 ubiquitin ligases (including the Apc) is that of a molecular scaffold. As the E3 binds both the E2 enzyme (ubiquitin conjugating enzyme) and the substrate, it brings specific lysine residues on the substrate into close proximity with an activated ubiquitin molecule (reviewed by Passmore & Barford, European Molecular Biology Organization Journal, 22(4): 786-796. (2004)). Additionally, Clute & Pines (1999) demonstrated that cyclin-B1 degradation occurs at the same location as Apc localization in HeLa cells.

The Apc indirectly triggers the degradation of cohesin, the protein complex that binds sister chromatids together. During Metaphase, sister chromatids are linked by intact cohesin complexes. The spindle checkpoint inhibits the Apc until all sister-kinetochores are attached to opposite poles of the mitotic spindle. When all kinetochores are properly attached, the spindle checkpoint is silenced and the Apc becomes active. The activated Apc then targets Securin for degradation. Securin inhibits a protease called separase, which cleaves cohesins allowing Anaphase onset.

### Cohesin

The replicated copies of each chromosome, the sister chromatids, are attached prior to their segregation in Mitosis and Meiosis. This association or cohesion is critical for each sister chromatid to bind to microtubules from opposite spindle poles, and thus segregate away from each other at Anaphase of Mitosis or Meiosis II. The cohesin protein complex is essential for cohesion in both Mitosis and Meiosis, and cleavage of one of the subunits is sufficient for loss of cohesion at Anaphase. The localization of the cohesin complex and other cohesion proteins permits evaluation of the positions of sister-chromatid associations within the chromosome structure, as well as the relationship between cohesion and condensation. A multisubunit complex called cohesin, contains Smc1p, Smc3p, Scc1p, and Scc3p, show here that Smc3p and a meiotic version of Scc1p called Rec8p are required for cohesion between sister chromatids, for formation of axial elements, for reciprocal recombination, and for preventing hyperresection of double-strand breaks during Meiosis. Both Rec8p and Smc3p colocalize with chromosome cores independently of synapsis during Prophase I and largely disappear from chromosome arms after pachytene but persist in the neighborhood of centromeres until the onset of Anaphase II. The eukaryotic cell's cohesion apparatus is required both for the repair of recombinogenic lesions and for chromosome segregation, and therefore appears to lie at the heart of the meiotic process. (Klein et al., Cell, 98(1): 91-103 (1999)).

### Rec8 Expression

Rec8 is a key component of the meiotic cohesin complex. During Meiosis, cohesin is required for the establishment and maintenance of sister-chromatid cohesion, for the formation of the synaptonemal complex, and for recombination between homologous chromosomes. We show that Rec8 has an essential role in mammalian Meiosis, in that Rec8 null mice of both sexes have germ cell failure and are sterile. In the absence of Rec8, early chromosome pairing events appear normal, but synapsis occurs in a novel fashion: between sister chromatids. This implies that a major role for Rec8 in mammalian Meiosis is to limit synapsis to between homologous chromosomes. In all other eukaryotic species studied to date, Rec8 phenotypes have been restricted to Meiosis. Unexpectedly, Rec8 null mice are born in sub-Mendelian frequencies and fail to thrive. These findings illuminate hitherto unknown Rec8 functions in chromosome dynamics during mammalian Meiosis and possibly in somatic development (Dev Cell. 8(6): 949-61 (2005)).

### Gamma Tubulin

The microtubule network, upon which transport occurs in higher cells, is formed by the polymerization of α (alpha)- and β (beta)-tubulin. The third major tubulin isoform, γ (gamma)-tubulin, is believed to serve a role in organizing this network by nucleating microtubule growth on microtubule-organizing centers, such as the centrosome. Research in vitro has shown that γ-tubulin must be restored to stripped centrioles to regenerate the centrosomal functions of duplication and microtubule nucleation. Fuller et al., Curr. Bio., 5(12): 1384-93 (1995) showed that the localization of γ-tubulin in isolated and in situ mammalian centrosomes using a novel immunocytochemical technique that preserves antigenicity and morphology while allowing increased accessibility. α-tubulin was localized in cytoplasmic and centriolar barrel microtubules and in the associated pericentriolar material. Foci of γ-tubulin were observed at the periphery of the organized pericentriolar material, as reported previously, often near the termini of microtubules. A further and major location of γ-tubulin was a structure within the proximal end of the centriolar barrel. The distributions were complementary, in that α-tubulin was excluded from the core of the centriole, and γ-tubulin was excluded from the microtubule barrel. γ-tubulin is localized both in the pericentriolar material and in the core of the mammalian centriole. Fuller suggests that γ-tubulin has a role in the centriolar duplication process, perhaps as a template for growth of the centriolar microtubules, in addition to its established role in the nucleation of astral microtubules.

### Pericentrin

Pericentrin is a highly conserved centrosome protein essential for cell division and microtubule organization. Pericentrin forms a large complex with γ-tubulin and other proteins involved in microtubule nucleation. Expression of mutated forms of pericentrin in cells induces the formation of ‘ectopic centrosomes’ that nucleate microtubules.

### Bora

Bora, a conserved protein that is required for the activation of Aurora A (AurA) at the onset of Mitosis.

### Polo Kinases

The highly conversed Polo kinase has been shown to regulate many aspects of mitosis. It promotes mitotic entry, centrosome duplication, spindle formation, removal of cohesin complexes from chromosomes, activation of the Anaphase Promoting Complex/Cyclosome (APC/C), mitotic exit, and cytokinesis. Polo kinase have also been shown to regulate meiosis. Lee, B. H. and Amon, A., “Polo Kinase: Meiotic Cell Cycle Coordinator,” Cell Cycle, 2 (5): 400-402 (2003); Descombes, P. and Nigg, E. A., “The Polo-Like Kinase Plx1 is Required for M Phase Exit and Destruction of Mitotic Regulators in Xenopus Egg Extracts,” The EMBO Journal, 17(5):1328-1335 (1998); and Bähler, J., et al., “Role of Polo Kinase and Midlp in Determining the Site of Cell Division in Fission Yeast,” J. Cell Biol., 143:1603-1616 (1998.

### Feo/Klp3A

Polo recruitment to the spindle midzone requires a complex formed by Fascetto (Feo) and Klp3A, the Drosophila homologue of KIF-4. Polo co-localizes with Feo and Klp3A and these two microtubule-associated proteins form a complex in vivo. D′Avino, P. P., et al., “Recruitment of Polo Kinase to the Spindle Midzone during Cytokinesis Requires the Feo/Klp3A Complex,” PLoS One, 6(e572): 1-8 (2007).

### INCENP

In human cells, the chromosome passenger proteins INner CENtromere Protein (INCENP), Aurora B, Borealin/Dasra B and Survivin exit in a complex termed the chromosomal passenger complex (CPC) and they are involved in regulating mitosis. Lens, S. M. A., et al., “Uncoupling the Central Spindle-Associated Function of the Chromosomal Passenger Complex From Its Role at Centromeres,” Mol. Biol. Cell, 17:1897-1909 (2006); Gassmann, R., et al., “Borealin: a Novel Chromosomal Passenger Required for Stability of the Bipolar Mitotic Spindle,” J. Cell Biol., 166:179-191 (2004); and Vader, G., et al., “The Chromosomal Passenger Complex Controls Spindle Checkpoint Function Independent from Its Role in Correcting Microtubule-Kinetochore Interactions,” Mol. Biol. Cell, 18:4553-4564 (2007).

### Spindle Checkpoint Factors

The core spindle checkpoint proteins are Mad1, Mad2, BubR1 (Mad3 in yeast), Bub1, Bub3 and Mps1. In cells containing disrupted spindles, the spindle assembly checkpoint arrests the cell cycle in Metaphase. The budding uninhibited by benzimidazole (Bub) 1, mitotic arrest-deficient (Mad) 1, and Mad2 proteins promote this checkpoint through sustained inhibition of the Anaphase-promoting complex/cyclosome. (Tunquist et al., J. Cell Biol., 163(6):1231-42 (2003)). The Mad and Bub proteins were first identified in budding yeast by genetic screens for mutants that failed to arrest in Mitosis when the spindle was destroyed (Taylor et al., 2004). These proteins are conserved in all eukaryotes. Several other checkpoint components, such as Rod, Zw10 and CENP-E, have since been identified in higher eukaryotes but have no yeast orthologues (Karess, 2005; Mao et al., 2003) (May and Hardwick, Journal of Cell Science, 119, 4139-4142 (2006)). CENP-E, found only in higher eurkaryotes, is a kinosin family member that binds to BubR1 and stimulates BubR1 kinase activity. BubR1 is required for capture and stabilization of microtubules at the kinetocore. Bub1 is a protein kinase that is important for recruiting other check point proteins.

### Activation of the Checkpoint

During Mitosis spindle microtubules bind to complex protein structures called kinetochores, which assemble on the centromere of each chromosome. The Mad and Bub proteins localize to the outer kinetochore early in Mitosis, before proper attachments are established, and accumulate on unattached kinetochores. When spindle microtubules make contact with the outer kinetochore, a number of complex molecular interactions take place that regulate both attachment and microtubule dynamics (Maiato et al., 2004). The checkpoint proteins are therefore ideally placed to monitor these interactions.

### Mei-332

The Drosophila mei-S332 gene acts to maintain sister-chromatid cohesion before Anaphase II of Meiosis in both males and females. The present invention relates to methods of cloning an animal by combining an activated oocyte with the genome from an activated donor cell.

### Cloning

“Cloning an animal” refers to producing an animal that develops from an oocyte containing genetic information or the nucleic acid sequence of another animal, the animal being cloned. The cloned animal has substantially the same or identical genetic information as that of the animal being cloned. “Cloning” also refers to cloning a cell, which includes producing an oocyte containing genetic information or the nucleic acid sequence of another animal. The resulting oocyte having the donor genome is referred to herein as a “nuclear transfer embryo.”

The present invention encompasses the cloning of a variety of animals. These animals include mammals (e.g., human, canines, felines), murine species (e.g., mice, rats), and ruminants (e.g., cows, sheep, goats, camels, pigs, oxen, horses, llamas). In particular, goats of Swiss origin, for example, the Alpine, Saanen and Toggenburg bread goats, were used in the Examples described herein. The donor cell and the oocyte are preferably from the same animal.

Both the donor cell and the oocyte are activated. An activated (e.g., non-quiescent) donor cell is a cell that is in actively dividing (e.g., not in a resting stage of Mitosis). In particular, an activated donor cell is one that is engaged in the mitotic cell cycle, such as G₁ phase, S phase or G₂/M phase. The mitotic cell cycle has the following phases, G₁, S, G₂ and M. The G₂/M phase refers to the transitional phase between the G₂ phase and M phase. The commitment event in the cell cycle, called START (or restriction point), takes place during the G₁ phase. “START” as used herein refers to late G₁ stage of the cell cycle prior to the commitment of a cell proceeding through the cell cycle. The decision as to whether the cell will undergo another cell cycle is made at START. Once the cell has passed through START, it passes through the remainder of the G₁ phase (i.e., the pre-DNA synthesis stage). The S phase is the DNA synthesis stage, which is followed by the G₂ phase, the stage between synthesis and Mitosis. Mitosis takes place during the M phase. If prior to START, the cell does not undergo another cell cycle, the cell becomes arrested. In addition, a cell can be induced to exit the cell cycle and become quiescent or inactive. A “quiescent” or “inactive” cell, is referred to as a cell in G₀ phase. A quiescent cell is one that is not in any of the above-mentioned phases of tile cell cycle. Preferably, the invention utilizes a donor cell that is a cell in the G₁ phase of the mitotic cell cycle. In certain methods described herein, Metaphase II cells are activated to enter Telophase II.

### Oocytes

Recent studies have explored the quality of oocytes for nuclear transfer. For example, the potential effect of both the genetic background and the maturation conditions on the Metaphase II (M-II) phenotype of mouse oocytes was studied. M-II oocytes from five different strains of mice were obtained either after superovulation (IVO) or by culture in a basal (IVM) or in a supplemented (IVM [symbol] ) maturation medium, and their phenotypic properties in terms of meiotic spindle size and organization, number of cytoplasmic microtubule organizing centers (MTOCs) and first polar body characteristics were compared. The results obtained reveal distinct phenotypic variations in the organization of the microtubule-centrosome complex based upon genetic background that are subjected to epigenetic changes during in vitro maturation.

### Induced Enucleation of Oocytes

Metaphase II oocytes are activated with 7% EtOH or 2 μM ionomycin, followed by treatment with Taxol (5 μg/mL), or cycloheximide (10 μg/mL) or demecolcine (0.4 μg/mL). resulting in the 2^{nd} polar body extrusion. In these studies, the control showed 3.7%: Taxol showed 3.6%, cycloheximide showed 16.3% and demecolcine showed 54%.

With demecolcine-induced enucleation, activated oocytes are treated with the microtubuline destabilizing drug, demecolcine. The resulting cytoplasts are competent to support term developments. Often, cytoplasts fail to complete the second polar body extrusion. Induced enucleation can take place, just prior to activation or just after activation.

### Oocyte Quality

Oocyte quality affects early embryonic survival, the establishment and maintenance of pregnancy, fetal development, and even adult disease. Quality, or developmental competence, is acquired during folliculogenesis as the oocyte grows, and during the period of oocyte maturation. Maintenance of oocyte quality is especially important in nuclear transfer methodology. As described herein, spindle associated factors aid in maintaining oocyte quality. The invention pertains to methods of maintaining these native factors within the oocyte during enucleation as well as potentially introducing exogenous spindle associated factors to improve oocyte quality and thus competence. This invention pertains processes occurring within the cytoplasm of the oocyte that are required for complete developmental competence.

### Cloning

It is preferable that the donor cells also be in the same stage of cell division. Using donor cells at certain phases of the cell cycle, for example, G₁ phase, allows for synchronization of the donor cells. One can synchronize the donor cells and put them in the same stage by depriving (e.g., reducing) the donor cells of a sufficient amount of nutrients in the media that allows them to divide. Once the donor cells have stopped dividing, then the donor cells are exposed to media (serum) containing a sufficient amount of nutrients to allow them to being dividing (e.g., Mitosis). The donor cells begin Mitosis substantially at the same time, and are therefore, synchronous. For example, the donor cells are deprived of a sufficient concentration of serum by placing the cells in 0.5% Fetal Bovine Serum (FBS) for about a week. Thereafter, the cells are placed in about 10% FBS and they will begin dividing at about the same time. They will enter the G1 phase about the same time, and are therefore, ready for the cloning process.

Methods of determining which phase of the cell cycle a cell is in are known to those skilled in the art, for example, U.S. Pat. No. 5,843,705 to DiTullio et al.; Campbell, K. H. S., et al., Embryo Transfer Newsletter, 14(1): 12-16 (1996); Campbell, K. H. S., et al., Nature, 380: 64-66 (1996); Cibelli, J. B., et al., Science, 280: 1256-1258 (1998); Yong, Z. and L. Yuqiang, Biol. of Reprod., 58: 266-269 (1998); and Wilmut, I., et al., Nature, 385: 810-813 (1997). As described below in the Examples, various markers are present at different stages of the cell cycle. Such markers can include cyclines D 1, 2, 3 and proliferating cell nuclear antigen (PCNA) for G_{1′} and BrDu to detect DNA synthetic activity. In addition, cells can be induced to enter the G₀ stage by culturing the cells on a serum-deprived medium. Alternatively, cells in G₀ stage can be induced to enter into the cell cycle, that is, at G₁ stage by serum activation (e.g., exposing the cells to serum after the cells have been deprived of a certain amount of serum).

The donor cell can be any type of cell that contains a genome or genetic material (e.g., nucleic acid), such as a somatic cell, germ cell or a stem cell. The term “somatic cell” as used herein refers to a differentiated cell. The cell can be a somatic cell or a cell that is committed to a somatic cell lineage. Alternatively, any of the methods described herein can utilize a diploid stem cell that gives rise to a germ cell in order to supply the genome for producing a nuclear transfer embryo. The somatic cell can originate from an animal or from a cell and/or tissue culture system. If taken from an animal, the animal can be at any stage of development, for example, an embryo, a fetus or an adult. Additionally, the present invention can utilize embryonic somatic cells. Embryonic cells can include embryonic stem cells as well as embryonic cells committed to a somatic cell lineage. Such cells can be obtained from the endoderm, mesoderm or ectoderm of the embryo. Embryonic cells committed to a somatic cell lineage refer to cells isolated on or after approximately day ten of embryogenesis. However, cells can be obtained prior to day ten of embryogenesis. If a cell line is used as a source for a chromosomal genome, then primary cells are preferred. The term “primary cell line” as used herein includes primary cells as well as primary derived cell lines.

Suitable somatic cells include fibroblasts (for example, primary fibroblasts), epithelial cells, muscle cells, cumulous cells, neural cells, and mammary cells. Other suitable cells include hepatocytes and pancreatic islets.

The genome of the somatic cell can be the naturally occurring genome, for example, for the production of cloned animals, or the genome can be genetically altered to comprise a transgenic sequence, for example, for the production of transgenic cloned animals, as further described herein.

Somatic cells can be obtained by, for example, disassociation of tissue by mechanical (e.g., chopping, mincing) or enzymatic means (e.g., trypsinization) to obtain a cell suspension, followed by culturing the cells until a confluent monolayer is obtained. The somatic cells can then be harvested and prepared for cryopreservation, or maintained as a stalk culture. The isolation of somatic cells, for example, fibroblasts, is described herein.

The nucleus of the donor cell is introduced before or upon exposure to the chemical or condition used to induce enucleation, or during any time prior cessation of protrusion of the second polar body containing essentially all of the endogenous chromatin. The donor nucleus and the enucleating oocyte can be combined in variety of ways to form the nuclear transfer embryo. For example, the genome of a donor cell can be injected into the activated oocyte by employing a microinjector (i.e., micropipette or needle). The nuclear genome of the donor cell, for example, a somatic cell, is extracted using a micropipette or needle. Once extracted, the donor's nuclear genome can then be placed into the activated oocyte by inserting the micropipette, or needle, into the oocyte and releasing the nuclear genome of the donor's cell. See, for example, McGrath, J. and D. Solter, Science, 226: 1317-1319 (1984).

Alternatively, the genome of a donor cell can be combined with an oocyte by fusion; e.g., electrofusion, viral fusion, liposomal fusion, biochemical reagent fusion (e.g., phytohemaglutinin (PHA) protein), or chemical fusion (e.g., polyethylene glycol (PEG) or ethanol). The nucleus of the donor cell can be deposited within the zona pelliduca which contains the oocyte. The steps of fusing the nucleus with the oocyte can then be performed by applying an electric field which will also result in a second activation of the oocyte. Anaphase II and/or Telophase II oocytes (e.g., oocyte having an extruding second polar body) used are already activated, hence any activation subsequent to or simultaneous with the introduction of genome from a somatic cell would be considered a second activation event. With respect to electrofusion, chambers, such as the BTX® 200 Embryomanipulation System, for carrying out electrofusion are commercially available from for example BTX®, San Diego. The combination of the genome of the donor cell with the oocyte results in a nuclear transfer embryo.

A nuclear transfer embryo of the present invention is then transferred into a recipient animal female and allowed to develop or gestate into a cloned or transgenic animal. Conditions suitable for gestation are those conditions that allow for the embryo to develop and mature into a fetus, and eventually into a live animal. Such conditions are known in the art. For example, the nuclear transfer embryo can be transferred via the fimbria into the oviductal lumen of each recipient animal female. In addition, methods of transferring an embryo to a recipient are known to those skilled in the art and are described in Ebert et al, Bio/Technology, 12: 69. “Cloning an animal” refers to producing an animal that develops from an oocyte containing genetic information or the nucleic acid sequence of another animal, the animal being cloned. The cloned animal has substantially the same or identical genetic information as that of the animal being cloned. “Cloning” also refers to cloning a cell, which includes producing an oocyte containing genetic information or the nucleic acid sequence of another animal. The resulting oocyte having the donor genome is referred to herein as a “nuclear transfer embryo.”

The present invention also relates to methods for generating transgenic animals. A transgenic animal is an animal that has been produced from a genome from a donor cell that has been genetically altered, for example, to produce a particular protein (a desired protein). Methods for introducing DNA constructs into the germ line of an animal to make a transgenic animal are known in the art. For example, one or several copies of the construct can be incorporated into the genome of a animal embryo by standard transgenic techniques.

Embryonal target cells at various developmental stages can be used to introduce transgenes. A transgene is a gene that produces the desired protein and is eventually incorporated into the genome of the activated oocyte. Different methods are used depending upon the stage of development of the embryonal target cell. The specific lines of any animal used to practice this invention are selected for general good health, good embryo yields, good pronuclear visibility in the embryo, and good reproductive fitness. In addition, the haplotype is a significant factor.

Genetically engineered donor cells for use in the instant invention can be obtained from a cell line into which a nucleic acid of interest, for example, a nucleic acid which encodes a protein, has been introduced.

A construct can be introduced into a cell via conventional transformation or transfection techniques. As used herein, the terms “transfection” and “transformation” include a variety of techniques for introducing a transgenic sequence into a host cell, including calcium phosphate or calcium chloride co-precipitation, DEAE dextrane-mediated transfection, lipofection, or electroporation. In addition, biological vectors, for example, viral vectors can be used as described below. Samples of methods for transforming or transfecting host cells can be found in Sambrook et al., Molecular Cloning: A Laboratory Manual In Second Edition, Cold Spring Harbor Laboratory, (Cold Spring Harbor Laboratory Press, Cold Spring Harbor, N.Y. 1989). Two useful and practical approaches for introducing genetic material into a cell are electroporation and lipofection.

The DNA construct can be stably introduced into a donor cell line by electroporation using the following protocol: donor cells, for example, embryonic fibroblasts, are resuspended in phosphate buffer saline (PBS) at about 4×10⁶ cells per mL. Fifty micrograms of linearized DNA is added to the 0.5 mL cell suspension, and the suspension is placed in a 0.4 cm electrode gap cuvette. Electroporation is performed using a BioRad Gene Pulser (Bio Rad) electroporator with a 330 volt pulse at 25 mA, 1000 microFarad and infinite resistance. If the DNA construct contains a neomyocin resistance gene for selection, neomyocin resistant clones are selected following incubation where 350 mg/mL of G418 (GIBCO BRL) for fifteen days.

The DNA construct can be stably introduced into a donor somatic cell line by lipofection using a protocol such as the following: about 2×10⁵ cells are plated into a 3.5 cm well and transfected with 2 mg of linearized DNA using LipfectAMINE® (GIBCO BRL). 48 hours after transfection, the cells are split 1:1000 and 1:5000 and if the DNA construct contains a neomyocin resistance gene for selection, G418 is added to a final concentration of 0.35 mg/mL. Neomyocin resistant clones are isolated and expanded for cyropreservation as well as nuclear transfer.

It is often desirable to express a protein, for example, a heterologous protein, in a specific tissue or fluid, for example, the milk of a transgenic animal. A heterologous protein is a protein that is not naturally made by the cloned species (e.g., a protein that is derived from a different species than the species being cloned). The heterologous protein can be recovered from the tissue or fluid in which it is expressed. For example, it is often desirable to express the heterologous protein in milk. Methods for producing a heterologous protein under the control of a milk-specific promoter is described below. In addition, other tissue-specific promoters, as well as, other regulatory elements, for example, signal sequences and sequences which enhance secretion of non-secreted proteins, are described below. The transgenic product (e.g., a heterologous protein) can be expressed, and therefore, recovered in various tissue, cells or bodily secretions of the transgenic animals. Examples of such tissue, cells or secretions are blood, urine, hair, skin, mammary gland, muscle, or viscera (or a tissue component thereof) including, but not limited to, brain, heart, lung, kidney, pancreas, gall bladder, liver, stomach, eye, colon, small intestine, bladder, uterus and testes. Recovery of a transgenic product from these tissues are well known to those skilled in the art. See, for example, Ausubel, F. M., et al., (eds), Current Protocols in Molecular Biology, vol. 2, ch. 10 (1991).

Useful transcriptional promoters are those promoters that are preferentially activated in mammary epithelial cells, including promoters that control the genes encoding protein such as caseins, β-lactoglobulin (Clark et al., Bio/Technology, 7: 487-492 (1989)), whey acid protein (Gordon et al., Bio/Technology, 5:1183-1187 (1987)), and lactalbumin (Soulier et al., Febs Letts., 297: 13 (1992)). Casein promoters can be derived from the α-, β-, γ-, or κ-casein genes of any animal species; a preferred promoter is derived from the goat β-casein gene (Ditullio, Bio/Technology, 10: 74-77 (1992)). Milk specific protein promoter or the promoters that are specifically activated in mammary tissue can be derived from cDNA or genomic sequences.

DNA sequence information is available for the mammary gland's specific genes listed above, in at least one, and often in several organisms. See, for example, Richards et al., J. Biol. Chem., 256: 526-532 (1981) (β-Lactalbumin rat); Campbel et al., Nucleic Acids Res., 12: 8685-8697 (1984) (rat WAP); Jones et al., J. Biol. Chem., 260: 7042-7050 (1985) (rat β-casein); Yu-Lee and Rosen, J. Biol. Chem., 258: 10794-10804 (1983) (rat β-casein); Hall, Bio. Chem. 1, 242: 735-742 (1987); (β-Lactalbumin human); Stewart, Nucleic Acids Res., 12: 389 (1984) (Bovine αS1 and α1 casein, cDNAs); Gorodetsky et al., Gene, 66: 87-96 (1988) (Bovine αβ-casein); Alexander et al., Eur. J. Biochem., 178: 395-401 (1988) (Bovine and β-casein); Brignon et al., Febs Let., 188: 48-55 (1977) (Bovine αS2 casein); Gamieson et al., Gene, 61: 85-90 (1987); Ivanov et al., Biol. Chem. Hopp-Seylar, 369: 425-429 (1988); Alexander et al., Nucleic Acid Res., 17: 6739 (1989) (Bovine β-Lactoglobulin); and Vilotte et al., Biochimie, 69: 609-620 (1987) (Bovine β-Lactalbumin).

The structure and function of the various milk protein genes are reviewed by Mercier & Vilotte, J. Dairy Sci., 76: 3079-3098 (1993). If additional flanking sequences are useful in optimizing expression of the heterologous protein, such sequences can be cloned using the existing sequences as probes. Mammary gland specific regulatory sequences from different organisms can be obtained by screening libraries from such organisms using known cognate nucleotide sequences, or antibodies to cognate proteins as probes.

Useful signal sequences, such as milk specific signal sequences or other signal sequences, which result in the secretion of eukaryotic or prokaryotic proteins, can be used. Preferably, the signal sequence is selected from milk specific signal sequences, that is, it is from a gene which encodes a product secreted into milk. Most preferably, the milk specific signal sequence is related to the milk specific promoter used in the construct. The size of the signal sequence is not critical. All that is required is that the sequence be of a sufficient size to effect secretion of the desired recombinant protein, for example, in the mammary tissue. For example, signal sequences from genes coding for caseins, for example α-, β-, γ- or κ caseins and the like can be used. A preferred signal sequence is the goat α-casein signal sequence. Signal sequences from other secreted proteins, for example, proteins secreted by kidney cells, pancreatic cells, or liver cells, can also be used. Preferably, the signal sequence results in the secretion of proteins into, for example, urine or blood.

A non-secreted protein can also be modified in such a manner that it is secreted such as by inclusion in the protein to be secreted all or part of the coding sequence of a protein which is normally secreted. Preferably, the entire sequence of the protein which is normally secreted is not included in the sequence of the protein but rather only a sufficient portion of the amino terminal end of the protein which is normally secreted to result in secretion of the protein. For example, a portion which is not normally secreted is fused (usually at its amino terminal end) to an amino terminal portion of the protein which is normally secreted.

In one aspect, the protein which is normally secreted is a protein which is normally secreted in milk. Such proteins include proteins secreted by mammary epithelial cells, milk proteins such as caseins, α-lactoglobulin, whey acid protein, and lactalbumin. Casein proteins including, α-, β-, γ- or κ-casein genes of any mammalian species. The preferred protein is α-casein, for example, goat α-casein. Sequences which encode the secreted protein can be derived from either cDNA or genomic sequences. Preferably, they are of genomic origin, and include one or more introns.

Other tissue specific promoters which provide expression in a particular tissue can be used. Tissue specific promoters are promoters which are expressed more strongly in a particular tissue than in others. Tissue specific promoters are often expressed exclusively in the specific tissue.

Tissue specific promoters which can be used include: a neural-specific promoter, for example, nestin, Wnt-1, Pax-1, Engrailed-1, Engrailed-2, Sonic-hedgehog: a liver specific promoter, for example, albumin, alpha-1, antitrypsin; a muscle-specific promoter, for example, myogenin, actin, MyoD, myosin; an oocyte specific promoter, for example, ZP1, ZP2, ZP3; a testus specific promoter, for example, protamine, fertilin, synaptonemal complex protein-1; a blood specific promoter, for example, globulin, GATA-1, porphobilinogen deaminase; a lung specific promoter, for example, surfactin protein C; a skin or wool specific promoter, for example, keratin, elastin; endothelium-specific promoter, for example, TIE-1, TIE-2; and a bone specific promoter, for example, BMP. In addition, general promoters can be used for expression in several tissues. Examples of general promoters, include β-actin, ROSA-21, PGK, FOS, c-myc, Jun-A, and Jun-B.

A cassette which encodes a heterologous protein can be assembled as a construct, which includes a promoter for a specific tissue, for example, for mammary epithelial cells, a casein promoter. The construct can also include a 3′ untranslated region downstream of the DNA sequence coding for the non-secreted proteins. Such regions can stabilize the RNA transcript of the expression system and thus increase the yield of desired protein from the expression system. Among the 3′ untranslated regions useful in the constructs for use in the invention are sequences that provide a polyA signal. Such sequences can be derived, for example, from the SV40 small t antigen, the casein 3′ untranslated region or other 3′ untranslated sequences well known in the art. In one aspect, the 3′ untranslated region is derived from a milk specific protein. The length of the 3′ untranslated region is not critical but the stabilizing effect of its polyA transcript appears imported in stabilizing the RNA of the expression sequence.

Optionally, the construct can include a 5′ untranslated region between the promoter and the DNA sequence encoding the signal sequence. Such untranslated regions can be from the same control region as that from which the promoter is taken or can be from a different gene, for example, they can be derived from other synthetic, semisynthetic or natural sources. Again, their specific length is not critical, however, they appear to be useful in improving the level of expression.

The construct can also include about 10%, 20%, 30% or more of the N-terminal coding region of a gene preferentially expressed in mammary epithelial cells. For example, the N-terminal coding region can correspond to the promoter used, for example, a goat α-casein N-terminal coding region.

The construct can be prepared using methods known to those skilled in the art. The construct can be prepared as part of a larger plasmid. Such preparation allows the cloning and selection of the correct constructions in an efficient manner. The construct can be located between convenient restrictions sites on the plasmid so that they can be easily isolated from the remaining plasmid sequences for incorporation into the desired animal.

Transgenic sequences encoding heterologous proteins can be introduced into the germ line of an animal or can be transfected into a cell line to provide a source of genetically engineered donor cells as described above. The protein can be a complex or multimeric protein, for example, a homo- or hetromultimeric proteins. The protein can be a protein which is processed by removing the N-terminus, C-terminus or internal fragments. Even complex proteins can be expressed in active form. Protein encoding sequences which can be introduced into the genome of an animal, for example, goats, include glycoproteins, neuropeptides, immunoglobulins, enzymes, peptides and hormones. The protein can be a naturally occurring protein or a recombinant protein for example, a fragment or fusion protein, (e.g., an immunoglobulin fusion protein or a mutien). The protein encoding nucleotide sequence can be human or non-human in origin. The heterologous protein can be a potential therapeutic or pharmaceutical agent such as, but not limited to, alpha-1 proteinase inhibitor, alpha-1 antitrypsin, alkaline phosphatase, angiogenin, antithrombin III, any of the blood clotting factors including Factor VIII, Factor IX, and Factor X chitinase, erythropoietin, extracellular superoxide dismutase, fibrinogen, glucocerebrosidas, glutamate decarboxylase, human growth factor, human serum albumin, immunoglobulin, insulin, myelin basic protein, proinsulin, prolactin, soluble CD 4 or a component or complex thereof, lactoferrin, lactoglobulin, lysozyme, lactalbumin, tissue plasminogen activator or a variant thereof. Immunoglobulin particularly preferred protein. Examples of immunoglobulins include IgA, IgG, IgE, IgM, chimeric antibodies, humanized antibodies, recombinant antibodies, single chain antibodies and anti-body protein fusions.

Nucleotide sequence information is available for several of the genes encoding the heterologous proteins listed above, in at least one, and often in several organisms. See, for example, Long et al., Biochem., 23(21): 4828-4837 (1984) (alpha-1 antitrypsin); Mitchell et al., Prot. Natl. Acad. Sci. USA, 83: 7182-7186 (1986) (Alkaline phosphatase); Schneider et al., Embo J., 7(13): 4151-4156 (1988) (Angiogenin); Bock et al., Biochem., 27 (16): 6171-6178 (1988) (Antithrombin); Olds et al., Br. J. Haematol., 78(3): 408-413 (1991) (Antithrombin III); Lyn et al., Proc. Natl. Acad. Sci. USA, 82(22): 7580-7584 (1985) (erythropoietin); U.S. Pat. No. 5,614,184 to Sytkowski et al. (erythropoietin); Horowtiz, et al., Genomics, 4(1): 87-96 (1989) (Glucocerebrosidase); Kelly et al., Ann. Hum. Genet., 56(3): 255-265 (1992) (Glutamate decarboxylase); U.S. Pat. No. 5,707,828 to Sreekrishna et al. (human serum albumin); U.S. Pat. No. 5,652,352 to Lichenstein et al. (human serum albumin); Lawn et al., Nucleic Acid Res., 9(22): 6103-6114 (1981) (human serum albumin); Kamholz et al., Prot. Matl. Acad. Sci. USA, 83(13): 4962-4966 (1986) (myelin basic protein); Hiraoka et al., Mol. Cell. Endocrinol., 75(1): 71-80 (1991) (prolactin); U.S. Pat. No. 5,571,896 to Conneely et al. (lactoferrin); Pennica et al., Nature, 301(5897): 214-221 (1983) (tissue plasminogen activator); and Sarafanov et al., Mol. Biol., 29: 161-165 (1995).

A transgenic protein can be produced in the transgenic cloned animal at relatively high concentrations and in large volumes, for example in milk, providing continuous high level output of normally processed protein that is easily harvested from a renewable resource. There are several different methods known in the art for isolation of proteins for milk.

Milk proteins usually are isolated by a combination of processes. Raw milk first is fractionated to remove fats, for example, by skimming, centrifugation, sedimentation, (H. E. Swaisgood, Development in Dairy Chemistry, I: Chemistry of Milk Protein, Applied Science Publishers, NY 1982), acid precipitation (U.S. Pat. No. 4,644,056 to Kothe et al.) or enzymatic coagulation with rennin or chymotrypsin (Swaisgood, ibid.). Next, the major milk proteins can be fractionated into either a clear solution or a bulk precipitate from which this specific protein of interest can be readily purified.

French Patent No. FR2487642 describes the isolation of milk proteins from skim milk or whey by performing ultra filtration in combination with exclusion chromatography or ion exchange chromatography. Whey is first produced by removing the casein by coagulation with rennet or lactic acid. U.S. Pat. No. 4,485,040 to Roger et al. describes the isolation of an α-lactoglobulin-enriched product in the retentate from whey by two sequential ultra filtration steps. U.S. Pat. No. 4,644,056 to Kothe et al. provides a method for purifying immunoglobulin from milk or colostrum by acid precipitation at pH 4.0-5.5, is sequential cross-flow filtration first on a membrane with 0.1-1.2 mm pore size to clarify the product pool and then on a membrane with a separation limit of 5-80 kD to concentrate it. Similarly, U.S. Pat. No. 4,897,465 to Cordle teaches the concentration and enrichment of a protein such as immunoglobulin from blood serum, egg yolks or whey by sequential ultra filtration on metallic oxide membranes with a pH shift. Filtration is carried out first at a pH below the isoelectric point (pI) of the selected protein to remove bulk contaminants from the protein retentate, next adding pH above the pI of the selected protein to retain impurities and pass the selected protein to the permeate. A different filtration concentration method is taught by European Patent No. EP467-482B1 in which defatted skim milk is reduced to pH 3-4, below the pI of the milk proteins, to solubilize both casein and whey proteins. Three successive rounds of ultra filtration are diafiltration and concentrate the proteins to form a retentate containing 15-20% solids of which 90% is protein. Alternatively, British Patent Application No. GB2179947 discloses the isolation of lactoferrin from whey by ultra filtration to concentrate the sample, fall by weak cation exchange chromatography at approximately a neutral pH. No measure of purity is reported in PCT Patent Publication No. WO 95/22258, where a protein such as lactoferrin is recovered from milk that has been adjusted to high ionic strength by the addition of concentrated salt, followed by cation exchange chromatography.

In all of these methods, milk or a fraction thereof is first treated to remove fats, lipids, and other particular matter that would foul filtration membranes or chromatography medium. The initial fractions can consist of casein, whey, or total milk protein, from which the protein of interest is then isolated.

PCT Patent Publication No. WO 94/19935 discloses a method of isolating a biologically active protein from whole milk by stabilizing the solubility of total milk proteins with a positively charged agent such as arginine, imidazole or Bis-Tris. This treatment forms a clarified solution from which the protein can be isolated, for example, by filtration through membranes that otherwise would become clogged by precipitated proteins.

Methods for isolating a soluble milk component, such as a peptide in its biologically active form, from whole milk or a milk fraction by tangential flow filtration are known. Unlike previous isolation methods, this eliminates the need for a first fractionation of whole milk to remove fat micelles, thereby simplifying the process in avoiding losses of recovery of bioactivity. This method can be used in combination with additional purification steps to further remove contaminants and purify the product (e.g., the protein of interest).

The following examples are intended to be illustrative and not limiting in any way. The nuclear transfer embryo can be maintained in a culture system until at least first cleavage (2-cell stage) up to the blastocyst stage, preferably the embryos are transferred at the 2-cell or 4-cell stage. Various culture media for embryo development are known to those skilled in the art. For example, the nuclear transfer embryo can be co-cultured with oviductal epithelial cell monolayer derived from the type of animal to be provided by the practitioner.

### EXEMPLIFICATION

### Example 1

### Materials and Methods

All animals were handled under the strict guidelines dictated by the Institutional Animal Care and Use Committee (IACUC) of Worcester Polytechnic Institute.

### Media Composition

FHM

| Working pH range | 7.2-7.4 |
|---|---|
| Components | mg/L |
| CaCl₂—2H₂O | 251.00 |
| KCL | 186.00 |
| KH₂PO₄ | 47.60 |
| MgSO₄ (anhyd.) | 24.10 |
| MgSO₄—7H₂O | 5550.00 |
| NaCl | — |
| NaHCO₃ | 336.00 |
| BSA | 1000.00 |
| EDTA | 3.80 |
| D-Glucose | 36.00 |
| HEPES | 4760.00 |
| Hyaluronidase (U/L) | — |
| Calcium Lactate | — |
| Sodium Lactate 60% (ml/L) | 1.86 |
| Lactate NaSalt (ml/L) 1.42 | — |
| Sodium Pyruvate | 22.00 |
| Phenol Red | 10.00 |
| L-Glutamine | 146.00 |
| Penicillin G Na Salt (u/L) | 100,000.00 |
| Streptomycin Sulfate | 50.00 |

KSOM

| Working pH range | 7.2-7.4 | 7.2-7.4 | 7.2-7.4 |
|---|---|---|---|
| Components | mg/L | mg/L | mg/L |
| CaCl₂—2H₂O | 250.00 | 250.00 | 250.00 |
| KCL | 186.38 | 186.38 | 186.38 |
| KH₂PO₄ | 47.99 | 47.99 | 47.99 |
| MgSO₄ | — | — | 0.00 |
| MgSO₄ 7H₂O | 49.30 | 49.30 | 49.30 |
| NaCl | 5551.80 | 5551.80 | 5551.80 |
| NaHCO₃ | 2100.25 | 2100.25 | 2100.25 |
| Other components | | | |
| EDTA | 3.72 | 3.72 | 3.72 |
| D-Glucose | 36.03 | 36.03 | 36.03 |
| Sodium Lactate | 1121.00 | 1121.00 | — |
| Lactate NaSalt (ml/L) | — | — | 1121.00 |
| 1.42 | | | |
| Sodium Pyruvate | 22.00 | 22.00 | 22.00 |
| BSA | 1000.00 | — | 1000.00 |
| Phenol Red | — | — | 10.00 |
| Amino acids | | | |
| L-Arginine | 63.20 | 63.20 | 63.20 |
| L-Cystine | 12.02 | 12.02 | 12.02 |
| L-Cystine-2HCL | — | — | 0.00 |
| L-Glutamine | 146.15 | 146.15 | 146.15 |
| Glycine | 3.75 | 3.75 | 3.75 |
| L-Histidine | — | — | — |
| L-Histidine•HCl•H₂O | 20.96 | 20.96 | 20.96 |
| L-Isoleucine | 26.23 | 26.23 | 26.23 |
| L-Leucine | 26.24 | 26.24 | 26.24 |
| L-Lysine | — | — | — |
| L-Lysine•HCl | 36.52 | 36.52 | 36.52 |
| L-Methionine | 7.46 | 7.46 | 7.46 |
| L-Phenylalanine | 16.52 | 16.52 | 16.52 |
| L-Serine | 5.26 | 5.26 | 5.26 |
| L-Threonine | 23.82 | 23.82 | 23.82 |
| L-Tryptophan | 5.11 | 5.11 | 5.11 |
| L-Tyrosine | 18.12 | 18.12 | 18.12 |
| L-Tyrosine NaH₂O | — | — | 0.00 |
| L-Valine | 23.42 | 23.42 | 23.42 |
| L-Alanine | 4.45 | 4.45 | 4.45 |
| L-Asparagine | — | — | — |
| L-Asparagine-H₂O | 7.50 | 7.50 | 7.50 |
| L-Aspartic Acid | 6.66 | 6.66 | 6.66 |
| L-Glutamic Acid | 7.36 | 7.36 | 7.36 |
| L-Proline | 5.76 | 5.76 | 5.76 |
| Antibiotics | | | |
| Pen G Na Salt (units) | 100,000.00 | 100,000.00 | 100,000.00 |
| Strep Sulfate | 50.00 | 50.00 | 50.00 |

### MTSB-XF

All chemicals purchased from Sigma Aldrich unless otherwise indicated.
% given in v/v in Phosphate Buffered Saline

| | | |
|---|---|---|
| PIPES | 100 | mM |
| MgCl2 | 5 | mM |
| EGTA | 2.5 | mM |
| DTT | 1 | mM |
| Taxol | 1 | uM |

| | |
|---|---|
| Aprotinin | 0.01% |
| Deuterium oxide | 50% |
| Formaldehyde | 3.70% |
| Triton X-100 | 0.10% |

### Blocking Buffer

All chemicals purchased from Sigma Aldrich unless otherwise indicated.
% given in w/v for solid and v/v for liquid chemicals in Phosphate Buffered Saline

| | |
|---|---|
| Sodium azide | 0.20% |
| Bovine Serum Abumin Fraction V | 1% |
| Powdered milk; Carnation | 0.2% |
| Normal Goat Serum (heat | 2% |
| inactivated) | |
| Glycine | 0.1M |
| Triton X-100 | 0.01% |

### Blocking Buffer (-Goat Serum)

All chemicals purchased from Sigma Aldrich unless otherwise indicated.
% given in w/v for solid and v/v for liquid chemicals in Phosphate Buffered Saline

| | |
|---|---|
| Sodium azide | 0.20% |
| Bovine Serum Abumin Fraction V | 1% |
| Powdered milk; Carnation | 0.2% |
| Glycine | 0.1M |
| Triton X-100 | 0.01% |

### Oocyte Collection

In order to induce superovulation in donor mice, female CF-1 mice (Charles River Laboratories) of breeding age were injected with Pregnant Mare Serum Gonadotropin (PMSG, Calbiochem) and Human Chorionic Gonadotropin (hCG, Calbiochem). For both hormones, 5IU was administered per mouse via intraperitioneal injection. PMSG was injected 64 hours before collection and hCG was given 48 hours later. Oviducts were dissected from mice euthanized by CO₂ asphyxiation and placed in FHM media (Chemicon) at 37° C. Oocytes were separated from surrounding cumulus cells by a brief exposure to bovine hyaluronidase (HA, Sigma, 150 units/ml, <10 minutes). Oocytes with poor morphology (lysed, fragmented, dark pigmentation) were discarded. Oocytes were washed three times in FHM media and randomly sorted into treatment groups. Some oocytes were immediately fixed (see below) at Metaphase of Meiosis II (MID.

### Oocyte Activation

Oocytes were activated with either a 5-minute incubation in 7% ethanol or a continuous exposure to 10 mM strontium chloride (SrCl₂, Sigma), and fixed at specific points in development.

For ethanol activation, all procedures were accomplished at 37° C. Denuded oocytes were washed 3 times in FHM and transferred to FHM containing 7% absolute ethanol. After 5 minutes, oocytes were washed 4 times with FHM, 3 times in KSOM (+aa, Chemicon), and incubated in KSOM at 37° C. in 5% CO₂. After 10 minutes, some oocytes were transferred to FHM containing 0.4 μg/mL demecolcine (Sigma).

For SrCl₂ activation, the denuded oocytes were washed 3 times in FHM, 3 to 4 times in KSOM (without Ca⁺², Chemicon) equilibrated to 37° C. in 5% CO₂, and then incubated in KSOM containing 10 mM strontium chloride at 37° C. with 5% CO₂. After 15 minutes, some oocytes, depending on experimental design, were transferred to KSOM containing both SrCl₂ (10 mM) and demecolcine (0.4 μg/mL) and incubated at 37° C. with 5% CO₂.

### Oocyte Fixation

Depending on experimental requirements, oocytes were either fixed at Metaphase of Meiosis II (MII) immediately following the FHM wash, or activated and fixed at t=25 minutes, t=125 minutes, t=245 minutes for Anaphase II, Telophase II, and Interphase respectively. The initial exposure to EtOH or SrCl₂ was considered T₀. For comparison purposes, oocytes were fixed in either 2% paraformaldehyde (PFA) solution containing 0.1% Triton X-100 or Microtubule Stabilization Buffer-Extraction Fixative (MTSB-XF); (Mattson et al., 1990)). Oocytes remained in fix solution for a minimum of 30 minutes at 37° C. and then were transferred to Blocking Buffer (block, Allworth & Albertini, Developmental Biology, 158: 101-112 (1993) for storage at 4° C.

### Oocyte Staining and Imaging

To localize Apc11, a polyclonal antibody raised in rabbits against N-terminal amino acids of human Apc11 (Santa Cruz) was used as a primary antibody. Oocytes were then washed 3 times with Phosphate Buffered Saline containing 0.1% Polyvinylpyrrolidone (PBS/PVP, Sigma) at room temperature and blocked with Blocking Buffer (block) for at least 30 minutes at room temperature. Apc11 was then probed with a goat anti-rabbit IgG antibody labeled with Alexa fluor 488 (5 μg/mL in Blocking Buffer, green, Molecular Probes) and extensively washed with PBS/PVP. Microtubules were localized using a 1:1 mixture of primary monoclonal antibodies raised against α-tubulin and β-tubulin (Sigma, 1:1000 dilution in Blocking Buffer), washed 3 times with PBS/PVP, blocked for at least 30 minutes with Blocking Buffer, and visualized with a goat anti-mouse IgG₁ secondary antibody labeled with Alexa fluor 594 (5 μg/mL in Blocking Buffer, red, Molecular Probes). Oocytes were subsequently washed with PBS/PVP and chromatin was visualized by exposure to Hoechst 22358 (10 μg/mL in block, blue, Molecular Probes). Oocytes were mounted on glass slides in 25 ul mounting solution (50% glycerol, 50% PBS, 25 mg/mL sodium azide), covered with cover glass (22×22 mm, #1, Fisher Scientific), and sealed with clear nail polish (New York Color Inc.). Imaging was accomplished on a Zeiss Axiovert 200M inverted fluorescence microscope coupled to a Roper CoolSnapFx camera through a 63× oil emersion objective and 10× eyepiece/camera lens. Metamorph and Axiovision image processing software was used to collect micrographs.

To visualize Cdc20, the protocol was similar to the visualization of Apc11 with different antibodies. The anti-Cdc20 antibody (Santa Cruz) was raised in rabbits against amino acids mapping to the N-terminal of human p55 (Cdc20). The secondary was a goat anti-rabbit IgG labeled with Alexa fluor 594 (5 μg/mL in Blocking Buffer, red, Molecular Probes). Since a red Alexa 594 secondary was used to label Cdc20, the tubulin secondary was switched to goat anti-mouse IgG₁ labeled with Alexa fluor 488 (5 μg/mL in Blocking Buffer, green, Molecular Probes).

For Rec8 staining, a similar procedure was followed. The polyclonal anti-Rec8 was raised in goats against amino acids mapping to the N-terminus of human Rec8 (Santa Cruz Biotech., sc-15152). The secondary was a donkey anti-goat IgG labeled with Alexa 594 (5 μg/mL, red, Molecular Probes). Tubulin visualization was accomplished using Alexa fluor 488 (5 μg/mL, green, Molecular Probes) as a secondary. To reduce tubulin staining, the αβ-tubulin cocktail was used at a 1:2000 dilution throughout the Rec8 staining protocol. In order to avoid non-specific binding, the blocking solution used throughout Rec8 staining contained no goat serum.

A minimum of 10 eggs were imaged for every treatment with each antibody. Unless otherwise stated, all images presented were representative of the group with little egg to egg variation.

### Antibody Optimization

Because the three primary antibodies have not been well characterized in mouse oocytes, it was first necessary to optimize the staining protocol. The same optimizing protocol was followed for each antibody.

### HeLa Cell Culture

The first step in the optimization process was to determine the localization pattern in HeLa cells. The HeLa cell culture was grown according to ATCC biosafety level 2 regulations in Minimum Essential Media, Eagle Salts (EMEM) with 10% Fetal Bovine Serum and penicillin/streptomycin at 37° C. under 5% CO₂. When cells were at or above 85% confluence, cultures were split 1:8. Cells were seeded on glass slides at 25% confluence, synchronized with a Thymidine/Hydroxyurea protocol according to Takita et al. (2003), and fixed in MTSB-XF for 1 hour at room temperature. Fixed cells were stored in Blocking Buffer at 4° C.

Synchronized and unsynchronized cells were stained for the presence and localization of Apc11, Cdc20, or Rec8. Initially, cells were incubated in varying concentrations of each primary antibody (1:1000, 1:500, 1:200) for 1 hour at room temperature. The cells were washed 2 times with PBS/PVP and incubated in the corresponding secondary antibody tagged with Alexa fluor 594 (5 μg/mL in Blocking Buffer) for 1 hour at room temperature. The cells were then washed again and subjected to a brief (˜15 minutes) incubation in Hoechst 22358 (1 μg/mL) to stain DNA. As negative controls, some cells were incubated in PBS in lieu of either primary or secondary antibody.

### Concentration Study

Once an ideal concentration was determined in HeLa cells, this information was used to optimize the staining protocol for mouse oocytes. All optimization studies were conducted with oocytes arrested at MII and randomly assorted into treatment groups. The same optimization procedure was followed for Apc11, Cdc20, and Rec8. Oocytes were incubated in one of several concentrations of primary antibody (1:100, 1:200, 1:500, 1:1000, 1:2000, 1:4000 in Blocking Buffer) before being imaged. Additionally, the incubation time and temperature was varied (1 hour at room temperature, 1 hour at 37° C., overnight at 4° C.). As negative controls, Blocking Buffer was substituted for either primary or secondary antibody for some oocytes. Following the incubations, the oocytes were imaged and the optimal protocol was determined (listed previously in Materials and Methods).

Following the concentration study, it was determined that an incubation in a 1:2000 dilution of anti-Apc11 overnight at 4° C. was optimal for Apc11 localization. For Cdc20, a 1-hour incubation at room temperature in 1:250 was ideal. For Rec8, a 1:100 dilution of primary antibody in Blocking Buffer was used.

### Results

### Part I: Antibody Validation

Since the localization of the Anaphase-promoting complex, specifically subunits Apc11 and Cdc20, has not been well studied in mouse oocytes, it was first necessary to validate the antibodies in a well characterized system like HeLa cells. Cells fixed in MTSB-XF were stained for either Apc11 or Cdc20 (red) and counterstained with Hoechst 22358. As a negative control, some cells underwent an identical staining procedure substituting blocking solution for the secondary antibody A-C) or primary antibody (D). Cells in negative control experiments appeared as dull, non-distinct red hazes. In non-dividing (G₂) cells, Apc11 appeared both cytoplasmically and in the nucleus A). However, the staining pattern was quite different in dividing cells. During Prophase, Apc11 began to bind the kinetochores (orange arrows). This staining then moved to the mitotic spindle during Metaphase (yellow arrow). This data is consistent with the report of Acquaviva et al. Nat Cell Biol. 6(9): 892-898 (2004), who demonstrated a similar localization for Apc3 (FIG. 5).

For Cdc20, the localization pattern is very similar. In non-dividing (G₂) cells, dim cytoplasmic and nuclear staining is detected. Once the cells enter Mitosis, the anti-Cdc20 is significantly more detectable around the dividing sister chromatids (yellow arrows). This is also consistent with previous work (FIG. 5) (Acquiviva et al., 2004, Clute & Pines, 1999).

The anti-Rec8 antibody was also initially validated with HeLa cells. Rec8 has been known to be a highly regulated protein (reviewed by Watanabe et al., 2005). It has been previously demonstrated that, while at Metaphase Rec8 is highly associated with the chromosomes, Rec8 is soon cleaved and disperses throughout the cytoplasm. This is similar to the localization pattern seen in the figures illustrating HeLa cells that were grown on coverglass to 75% confluence and fixed in 2% PFA with Trition X-100. Cells were stained for Rec8 (red) and chromatin (blue). Non-replicative cells appear show a non-distinct cytoplasmic Rec8 staining. However, the cell at Metaphase (yellow arrow) shows bright Rec8 staining around the chromosomes.

### Part II: Optimization

### Dilution Study

Once the antibodies were validated, it was then necessary to optimize the staining protocol in a mouse oocyte system. In order to determine the ideal experimental conditions, staining variables such as primary antibody concentration, incubation temperature, and duration all needed to be addressed. In brief, a series of experiments was designed such that primary concentration, incubation time, and temperature were individually varied. A similar process was completed for the optimization of Apc11, Cdc20 (data not shown) and Rec8 (data not shown). The detailed experimental design is provided in the Methods and Materials section.

FIG. 7 shows representative results of negative control experiments. To generate these images, oocytes were subjected to the same staining protocol listed in the Materials and Methods section without the addition of secondary antibody (FIGS. 7B-D). FIG. 7A shows an egg stained with neither primary nor secondary antibodies. In all oocytes, low levels of non-distinct staining could be detected. It was this base level of fluorescence to which all subsequent images were compared.

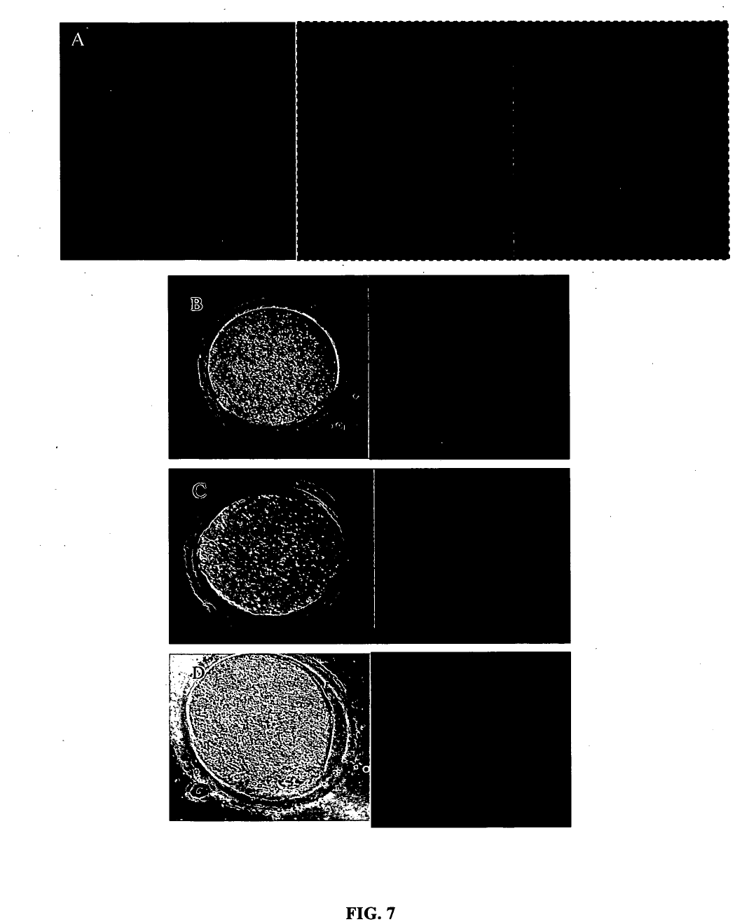

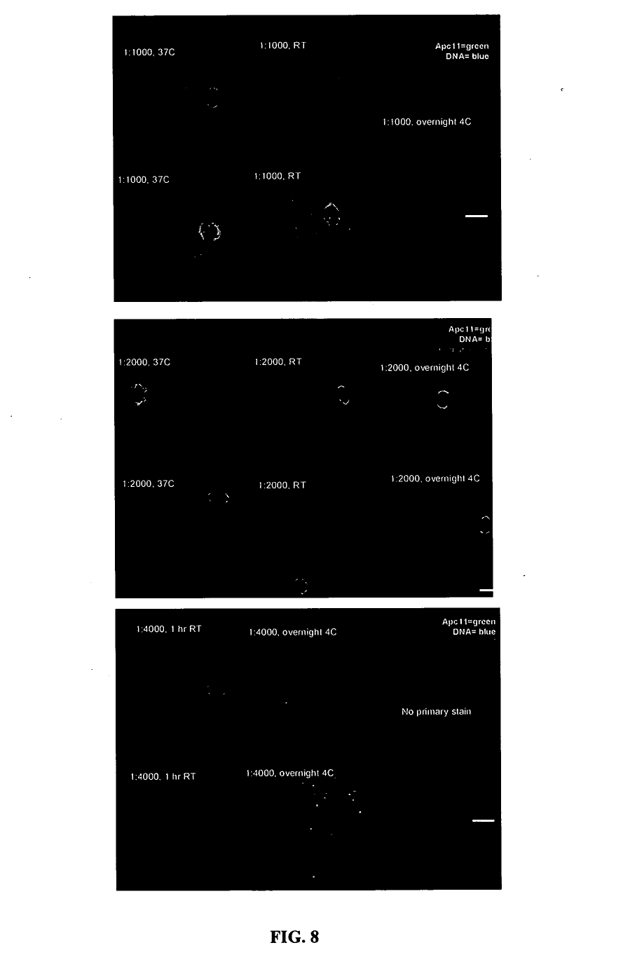

The pictures in FIG. 8 are representative of the optimization study for Apc11. Oocytes were fixed at Metaphase of Meiosis II in 2% PFA with Triton X-100. Apc11 appears in green. Chromatin stained with Hoechst 22358 appears blue. Tubulin (meiotic spindle) is stained red. In samples incubated in high concentrations of anti-Apc11 (every dilution tested below 1:1000), the staining pattern was that of complete saturation. Camera saturation occurs when pixel values exceed the range of the camera and are assigned as white.

At high concentrations, it was impossible to differentiate any variation within a single oocyte or between other oocytes (data not shown). At the 1:1000 dilution (FIG. 8, top panel), distinct Apc11 localization patterns began to appear within the oocyte (cytoplasmic staining, cortical omission, and an exclusion zone within the meiotic spindle, detailed later). As the dilution was increased to 1:2000 (FIG. 8, middle panel), these patterns became more consistently apparent. While these patterns were still detectable in the 1:4000 dilution (FIG. 8, bottom panel), the staining pattern was often so dim, that it was difficult to discern the antibody from the background auto-fluorescence of the oocyte (shown in FIG. 7). It was therefore determined that a 1:2000 dilution of anti-Apc11 was ideal for the purposes of this project.

For this experimental set, secondary antibody conditions were held constant at 5 μg/mL.

### Incubation Temperature and Duration

Once a dilution was selected, it was then necessary to determine the optimal conditions for temperature and duration of immunostaining. Oocytes were incubated overnight at 4° C. or for 1 hour at either 37° C. or room temperature. While oocytes stained at 37° C. yielded bright images (FIG. 8, middle panel-left), often the signal reported by the camera was saturated and therefore may not have been as specific as the pictures taken of oocytes incubated at lower temperatures. In contrast to this, oocytes incubated for 1 hour at room temperature (FIG. 8, middle panel-middle), were often too dim to discern any consistent localization. Similarly, oocytes stained at 4° C. overnight (FIG. 8, middle panel-right) were also fairly dim, but localization within these oocytes appeared more consistent than those stained at room temperature. Because of this, an overnight incubation at 4° C. was used for the remainder of the Apc11 studies.

### Fixative Comparison

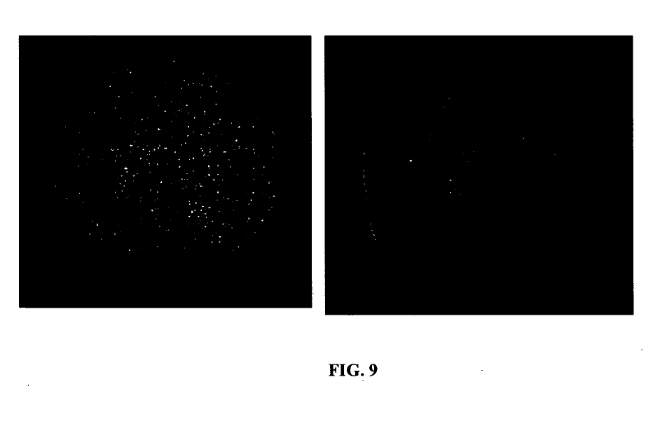

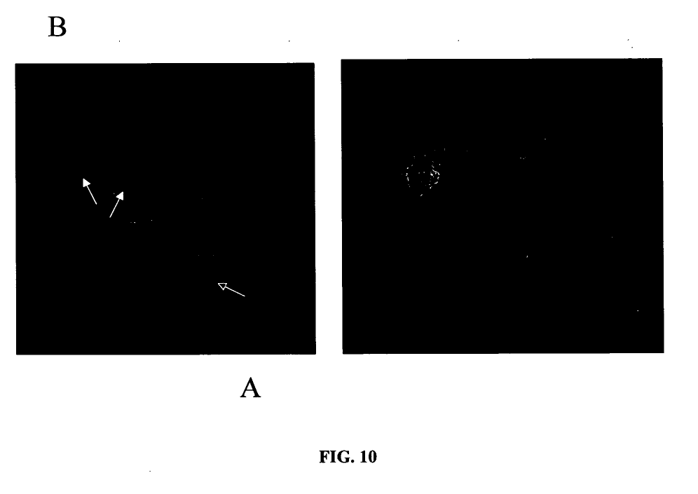

Another factor that has a profound effect on staining specificity and the imaging process is the solution used to fix the oocytes. Ideally, the fixative should preserve the structure of certain aspects of an immobilized cell in order to help predict the utility of those aspects in vivo. For example, if conducting studies on the meiotic spindle, a fix solution that would preserve the native microtubule structure at a given time while simultaneously removing material that would restrict access to the spindle would be ideal. In the mouse system, one such fix is the Microtubule Stabilizing Buffer-Extraction Fixative (MTSB-XF) used by (Ibanez et al., 2003) who carefully measured the morphology of the meiotic spindle in response to a variety of stimuli. Since the Anaphase-promoting complex was known to act in the vicinity of the meiotic spindle, MTSB-XF was chosen as the fix solution for the initial studies of the Apc11 antibody. Representative results of these initial studies are shown in FIG. 8. Oocytes arrested at MII were stained with Anti-Apc11 (green), Hoechst 22358 (blue), and Texas-red Phalloidin (red). Although an Apc11 localization pattern can be detected as an exclusion zone surrounding the meiotic spindle, the overall stain is hazy and non-distinctive. For this reason, MTSB-XF was replaced by the PFA solution described in the Methods and Materials section as the preferred fixative (compare FIG. 9 to FIG. 10).

### Selection of an Activation Stimulus

As oocytes develop, they undergo a complete round of Meiosis and then arrest at Metaphase of Meiosis II. Oocytes will remain in this arrested state until they are ionically activated to continue development. Normally, this stimulus is a periodic calcium signal produced by the invading sperm to the oocyte. However, in order to study the spatial localization of the Anaphase-promoting complex at different stages of development, this signal was initially replicated in vitro with a short incubation in 5% Ethanol. Ethanol causes the formation of inositol 1,4,5-triphosphate at the membrane and a concomitant influx of extra-cellular calcium (Ibanez et al., 2005). However, instead of periodic spikes in cytoplasmic calcium concentration, the ethanol causes a prolonged influx of calcium. As a result, oocytes activated with ethanol developed inconsistently. Often, as many as 50% of eggs per experiment failed to leave MII when activated by a standard ethanol protocol (data not shown). Additionally, eggs that did activate often progressed through development too quickly for the cell to properly respond. Since this phenomenon often caused significant egg-to-egg variation in control groups (data not shown), ethanol was replaced as an activation stimulus by strontium chloride for all subsequent experiments.

### Part III: Apc11 Localization

Demecolcine has been used to aid in the enucleation process for the purposes of somatic cell nuclear transfer (Baguisi & Overstrom, 2000). In order to test the effects of demecolcine on the spatial localization of Apc11, the catalytic core of the Anaphase-promoting complex, oocytes harvested from hormonally primed CF-1 mice were activated in strontium chloride, incubated in the presence of 0.4 μg/mL demecolcine, and fixed in PFA solution at specific points of development (AII, TII, Interphase). Control eggs were fixed without ever being exposed to demecolcine. The results of these control experiments can be found in FIG. 10 through FIG. 13. Anti-Apc11 is shown in green. Chromatin appears blue. Tubulin appears red. Left panels show Apc11 alone; right panels are the overlay of the three stains. A minimum of 10 eggs were imaged for every treatment with each antibody. Unless otherwise stated, all images presented were representative of the treatment group with little egg-to-egg variation.

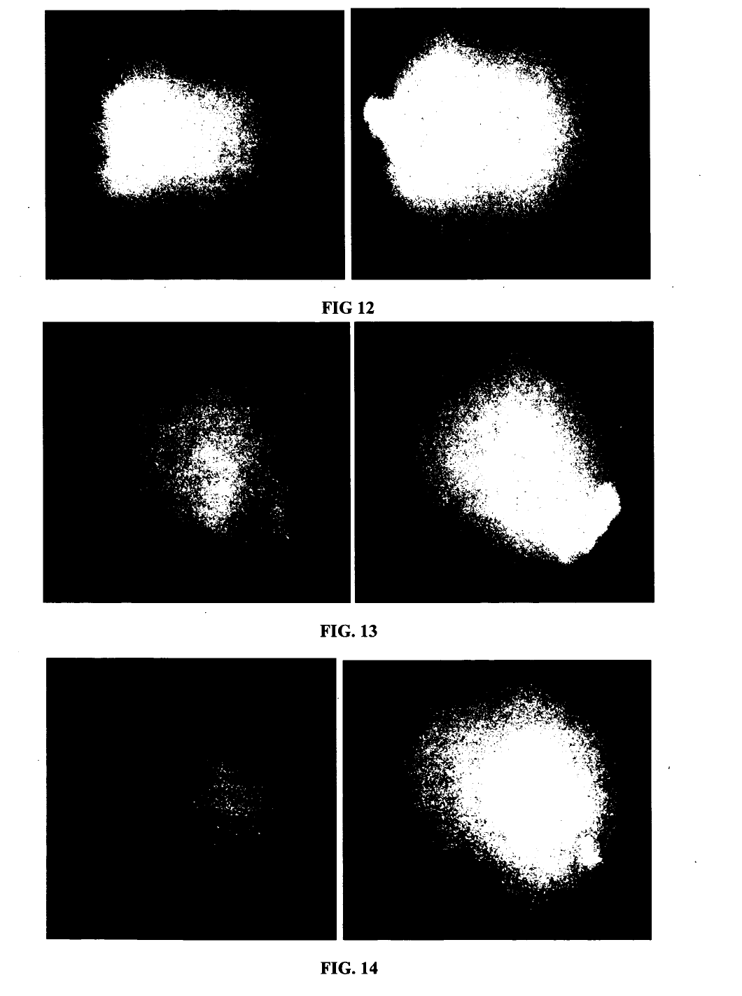

During Meiosis II (MID, Apc11 (green) shows two types of localization. The most prevalent is a strong localization to the area directly surrounding the meiotic spindle FIG. 10, (yellow arrows). This perispindular localization persists through all phases of Meiosis II and begins to disappear at the onset of interphase (FIG. 13). Interestingly, while there exists a high concentration of Apc11 outside the spindle, there is very little staining in the area directly within the spindle (not shown in the focal plane of FIG. 10; see FIG. 12). The second type of localization occurs only at MII and early AII. Within these oocytes, there appears to be a discrete staining pattern within the hemisphere that contains the meiotic spindle (FIG. 10, orange arrow).

### Effects of Demecolcine on Apc11 Spatial Localization

Once the spatial localization of Apc11 following a standard parthenogenetic activation was established, it was then possible to determine what effects demecolcine may have on its localization. Oocytes were incubated 10 minutes in strontium chloride before they were transferred into media containing both strontium chloride and demecolcine (0.4 μg/mL). The results of these experiments can be found in FIG. 14 through FIG. 16. Anti-Apc11 is shown in green. Chromatin appears blue. Tubulin appears red. Left panels show Apc11 alone; right panels are the overlay of the three stains.

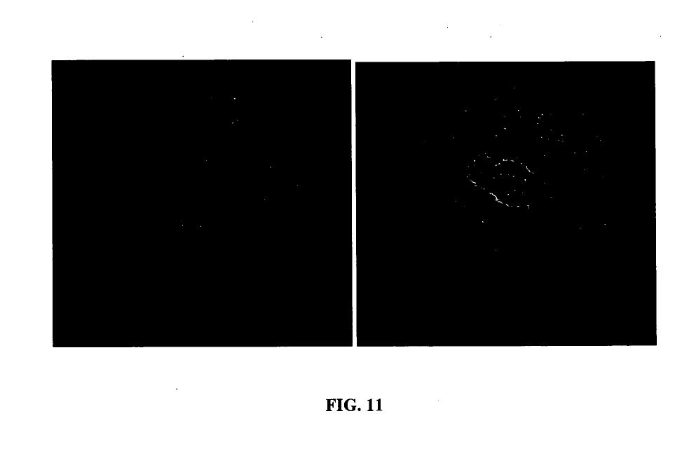

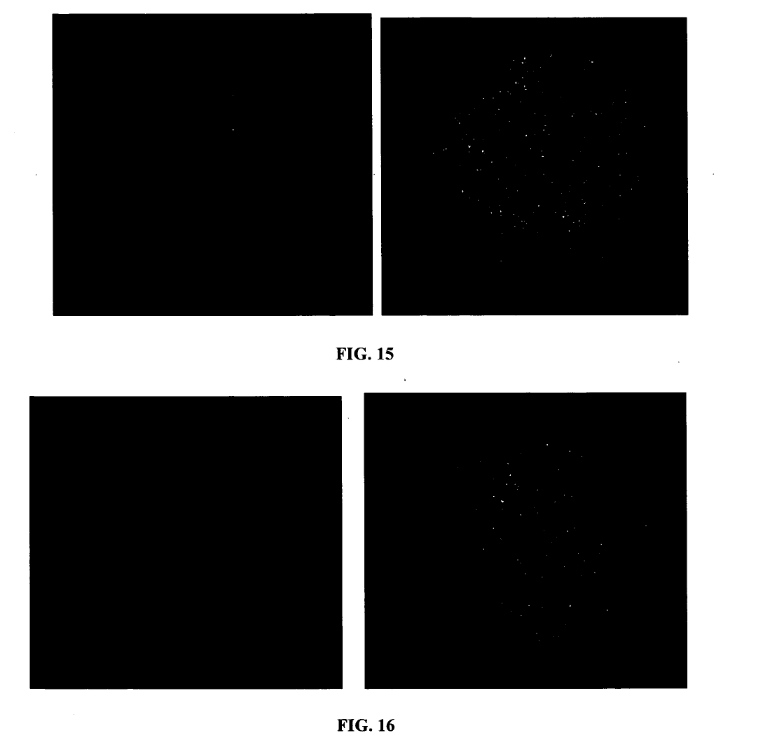

As seen in FIG. 14 the demecolcine destabilizes the microtubules within the cell. As a result, the meiotic spindle is severely disrupted compared to control eggs at the same time (see FIG. 11 and tubulin (red staining)) is detected throughout the cytoplasm. Furthermore, a longer incubation in demecolcine causes more of the microtubules to disassociate from the spindle (compare red staining in FIG. 14 to FIG. 16). Because the spindle was disrupted, sister chromatids often did not segregate properly and only a single cluster of DNA was observed well after the activation stimulus. Eventually, the oocyte completely extrudes its chromatin in the second polar body (FIG. 16).

Since the spatial localization of many key cell cycle proteins is closely associated with the meiotic spindle, it was hypothesized that the disruption of that spindle could negatively affect subunits of the Apc as well. As FIG. 14 through FIG. 16 show, the disruption of the meiotic spindle did cause a concomitant loss of Apc11 localization. Apc11 localization in mouse eggs is characterized by an aggregation of protein directly around the spindle. However, in eggs treated with demecolcine, the staining pattern changed to a non-distinct ataxia across the entire oocyte with no evidence of colocalization in any stage of development.

### Part IV: Cdc20 Localization

In the regulation of development, Cdc20 has the dual role of both an activator of the Anaphase-promoting complex and a substrate of the ubiquitin-assisted destruction pathway. During Metaphase of Mitosis, Cdc20 binds to the Apc allowing for the ubiquitination of Securin. Once the cell progresses beyond early Anaphase, Cdc20 disassociates from the complex and is soon destroyed. In order to determine if this pattern in Mitosis correlates to meiotic cells, Cdc20 was localized in mouse eggs with a commercially available polyclonal antibody.

### Control Activation

Oocytes were harvested from the oviducts of hormonally primed CF-1 mice and separated from the surrounding cumulus mass with bovine hyaluronidase (HA). Denuded oocytes were either fixed immediately at Metaphase of Meiosis II or activated with 10 mM strontium chloride and fixed later in development in 2% PFA with Triton X-100 (AII, TII, Interphase). Oocytes were stained for Cdc20 (red), chromatin (blue), and αc+β tubulin (green).

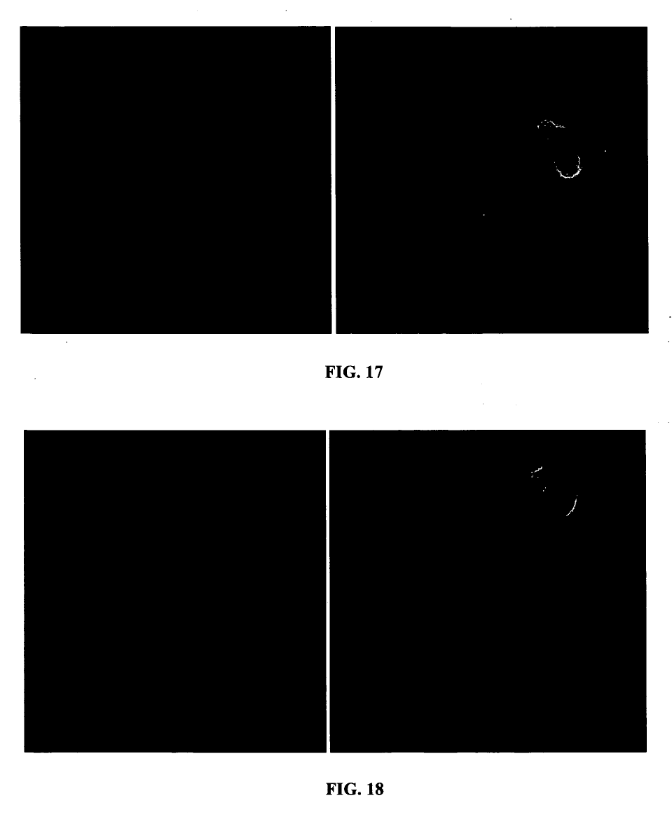

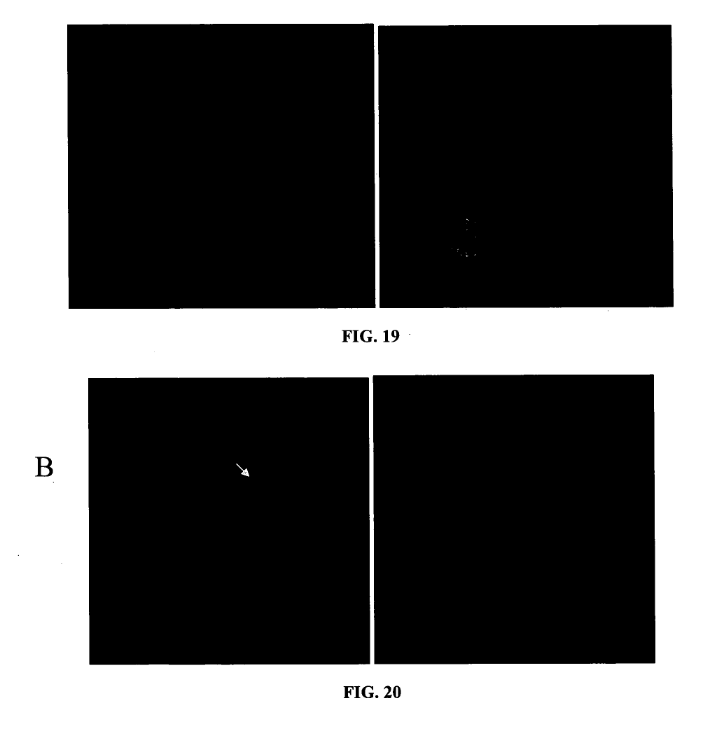

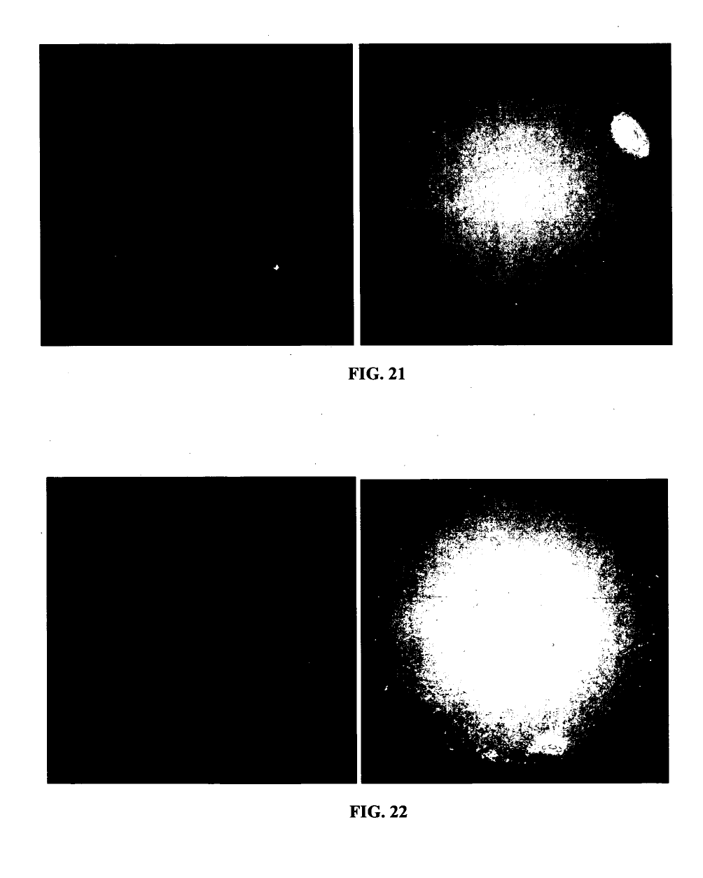

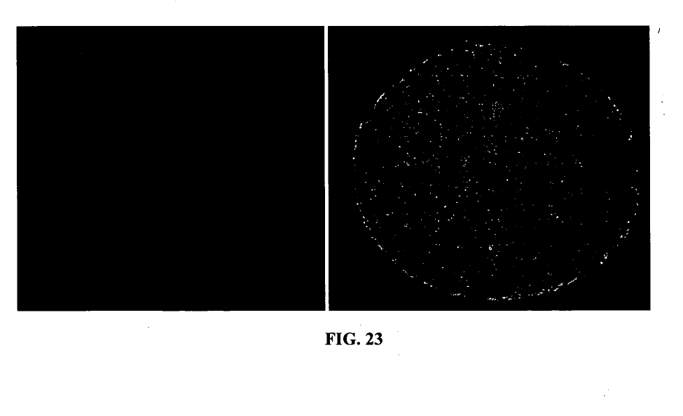

At MII, Cdc20 staining (red) appears as punctate spots seemingly randomly distributed throughout the cytoplasm. Therefore, unlike Apc11, Cdc20 does not appear to localize to the vicinity of the meiotic spindle in Metaphase. This variegate staining disappears early after activation. At AII, the staining pattern has changed to a more diffuse cytoplasmic distribution across the cell (see FIG. 18). By Telophase II (TII, Cdc20 staining has all but disappeared, with only a faint haze remaining across the cytoplasm. This miasma is indistinguishable from the natural autofluorescence of oocytes fixed with PFA (data not shown). Interestingly, Cdc20 staining reappears in oocytes fixed in Interphase (FIG. 19). At this point in development, Cdc20 localizes strongly to the pronucleus (yellow arrow) and diffusely to the cytoplasm. This cytoplasmic staining is comparable to the staining observed at AII (compare FIG. 14 to FIG. 18).

### Effects of Demecolcine on Cdc20 Localization

In order to determine if the localization of Cdc20 is affected by an incubation in demecolcine, oocytes harvested from hormonally primed CF-1 mice were fixed at various stages of development in the presence or absence of demecolcine and stained for Cdc20 (red), α+β tubulin (green), chromatin (blue).

Similar to observations with Apc11, the demecolcine caused a severely disrupted spindle and the associated cytoplasmic distribution of tubulin, and as the demecolcine incubation duration was increased, these effects became more prevalent. Also, just as Apc11 localization was disturbed by spindle destruction of demecolcine, so too was Cdc20 affected. In control eggs, Cdc20 staining levels varied greatly throughout development, nearly disappeared during TII. As seen in eggs incubated with demecolcine showed low levels of muddled signal across the cytoplasm. This pattern persisted throughout development.

### Part V: Rec8 Localization

With the knowledge that demecolcine can affect both the spatial localization of Apc11, the catalytic core of the Anaphase-promoting complex, and the orderly destruction of Cdc20, a main activator of the Apc, one can postulate that the activity of the Apc could also be affected by an incubation in demecolcine. In order to determine the magnitude of such an effect, the spatial localization of Rec8 was examined.

Rec8 is a meiosis-specific subunit of the cohesion complex. In order for a cell to leave Metaphase, the Apc driven disassembling of the cohesion complex surrounding sister chromatids must occur. The Apc, activated by Cdc20, ubiquitinates Securin marking it for destruction by the 26S proteasome. The destruction of Securin activates Separase to open the cohesion complex by the cleavage of the subunit Rec8. Therefore, the spatial localization of Rec8 in relation to cellular chromatin could potentially serve as an indirect measure of downstream Apc activity.

### Control Activation Experiments

Since Rec8 localization has not been well characterized in mouse oocytes, it was first necessary to determine the staining pattern in oocytes fixed and activated with a standard parthenogenetic protocol. Denuded oocytes were harvested from hormonally primed CF-1 mice and fixed in 2% PFA with Triton X-100 either immediately after removal from hyaluronidase or activated with 10 mM strontium chloride and fixed later in development. They were then stained for chromatin (blue) with Hoechst 22358 and Rec8 (red) with a commercially available Donkey anti-goat polyclonal antibody (Santa Cruz Biotechnologies).

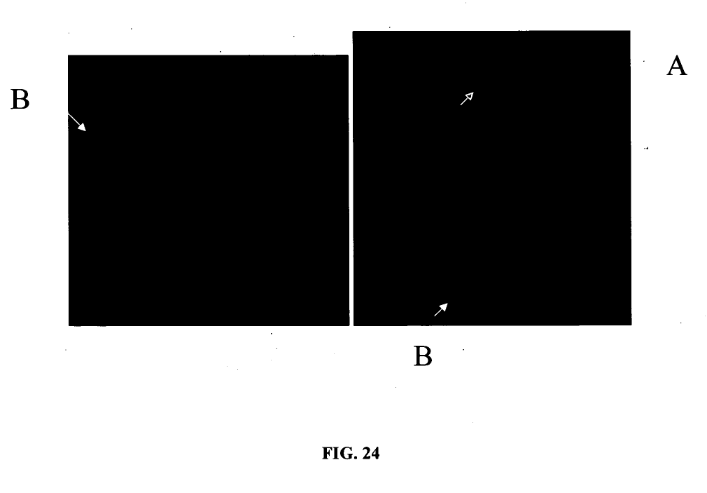

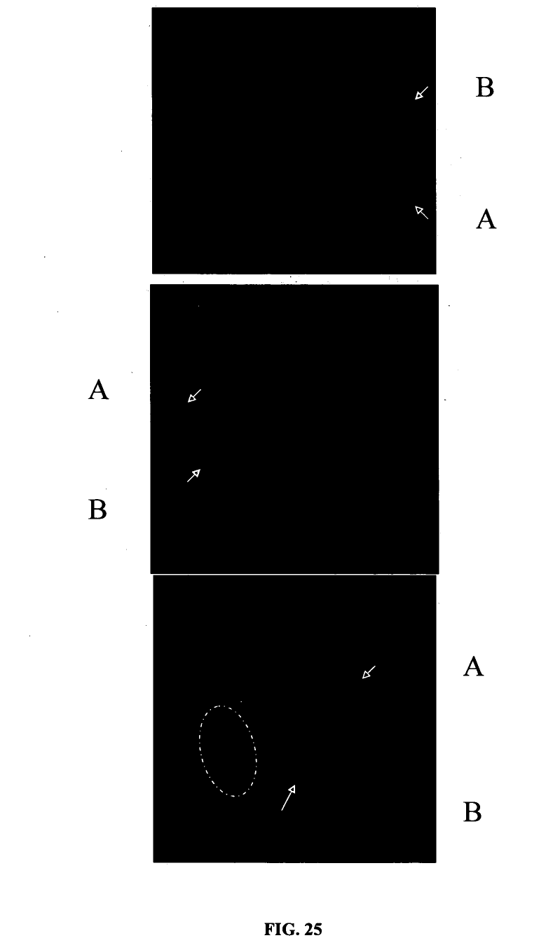

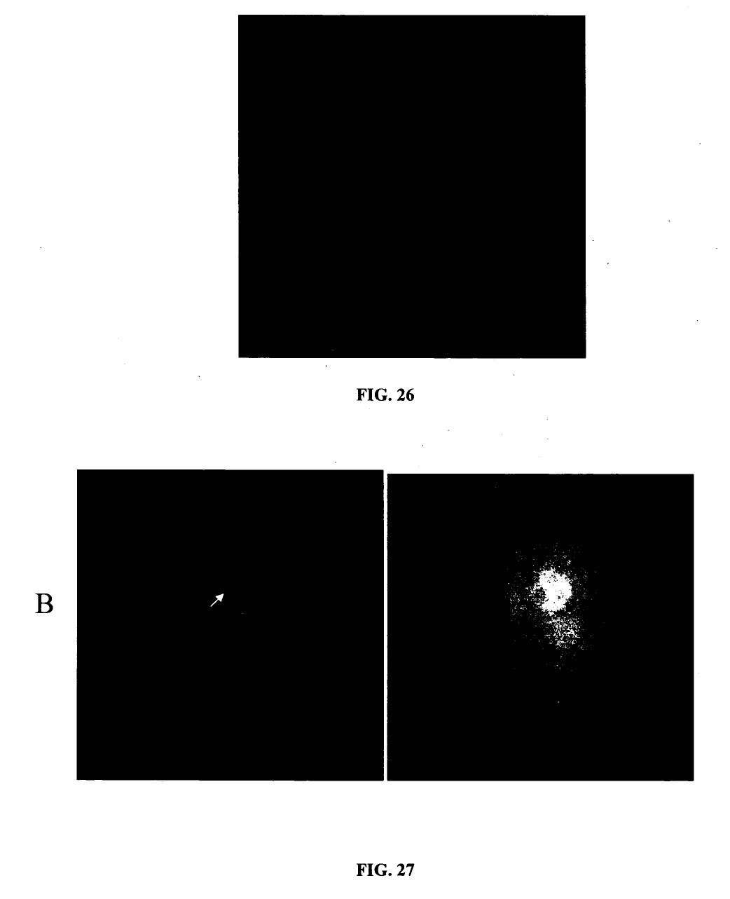

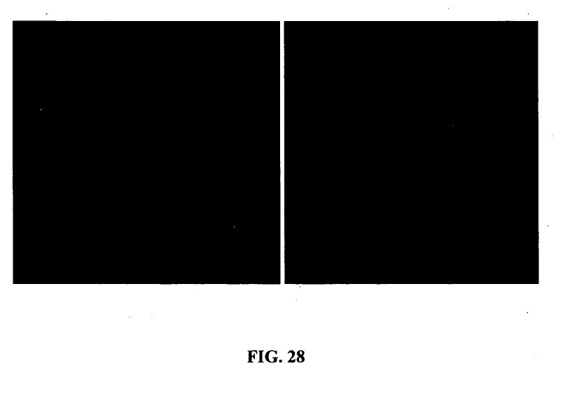

Anti-Rec8 staining appears to localize to the vicinity of cellular chromatin in several ways. Firstly, Rec8 localizes to the membrane surrounding the first polar body (FIG. 24, right panel, orange arrow; and FIG. 25, bottom panel, aqua circle). This staining can be seen in all oocytes in which the polar body contains distinguishable chromatin. However, in oocytes where the chromatin within the polar body has begun to deteriorate, there is no evidence of Rec8 polar body localization (data not shown).

Secondly, during MII, Rec8 is sequestered to the cortical region directly overlying the metaphase plate (FIG. 24; yellow arrows). As the sister chromatids begin to separate early in AII, this cortical staining splits as well (FIG. 25; top panel) remaining closely tied to both sets of chromosomes. As Anaphase progresses into Telophase (FIG. 25; middle and bottom panels), it becomes apparent that Rec8 localizes to both the female pronucleus and the budding second polar body. This localization continues though Telophase II (FIG. 27; yellow arrow).
The Effects of Demecolcine on Rec8 Localization Incubation in Demecolcine-Affected Rec8 Localization within Mouse Oocytes

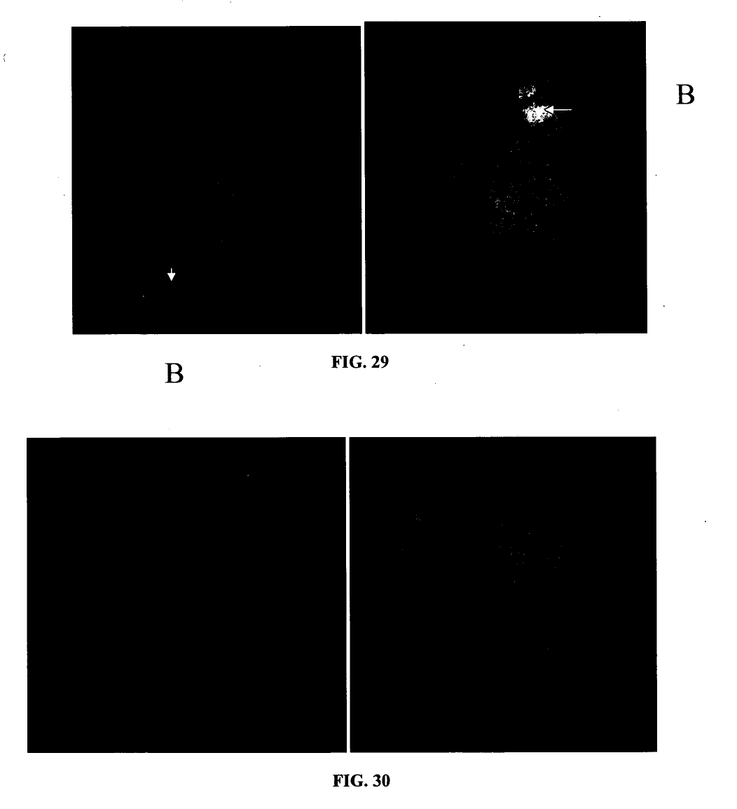

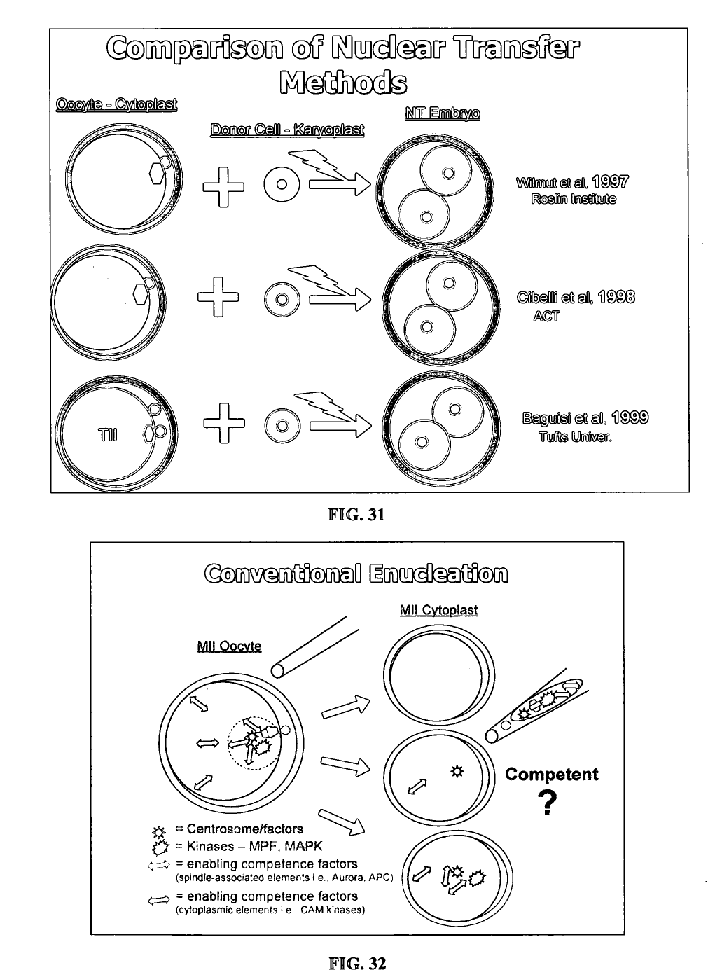

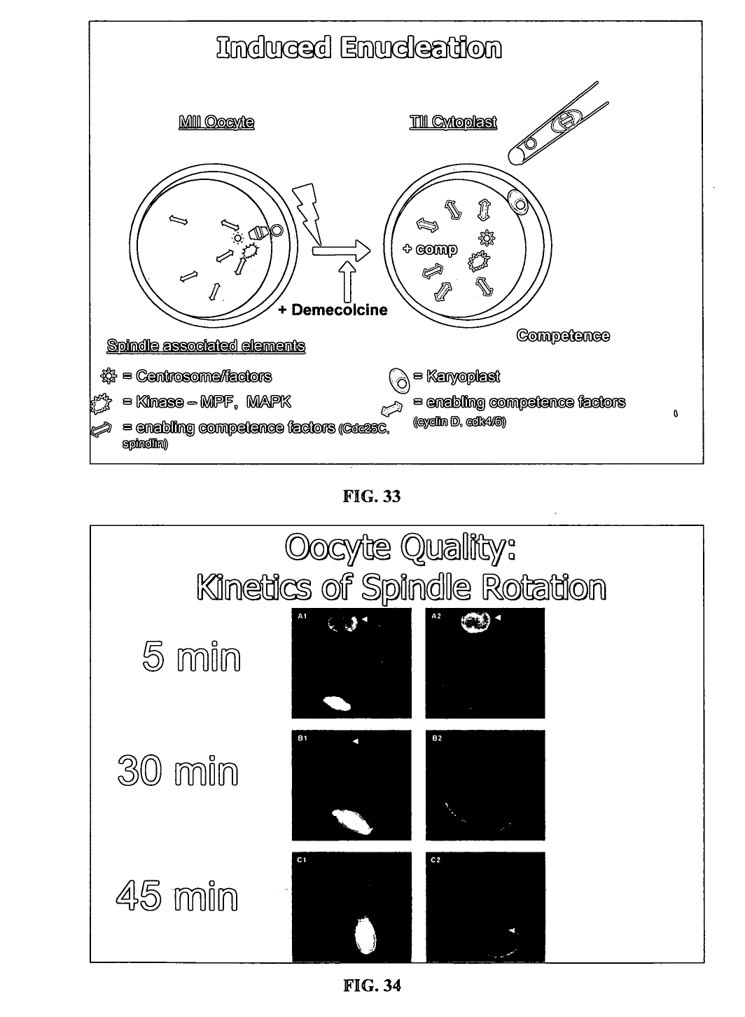

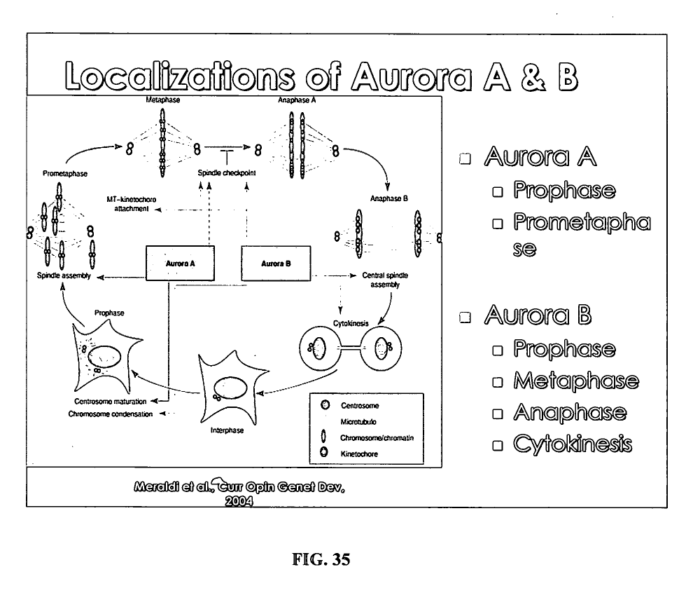

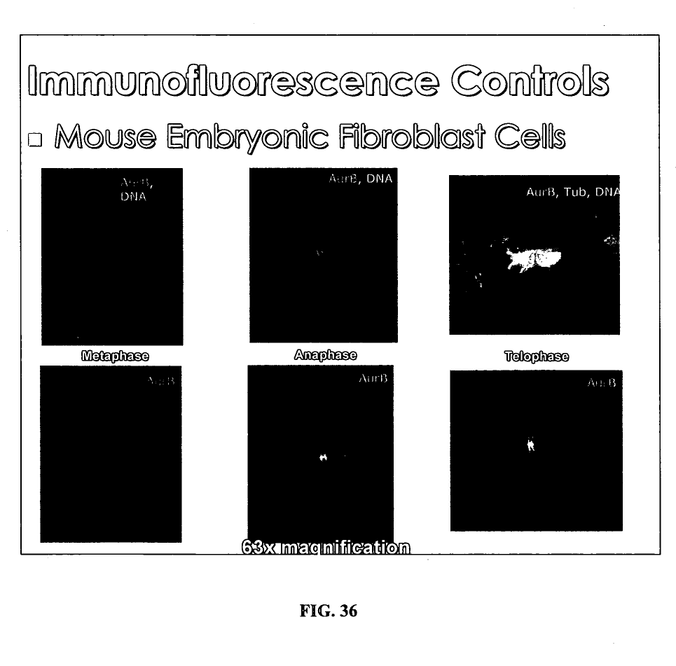

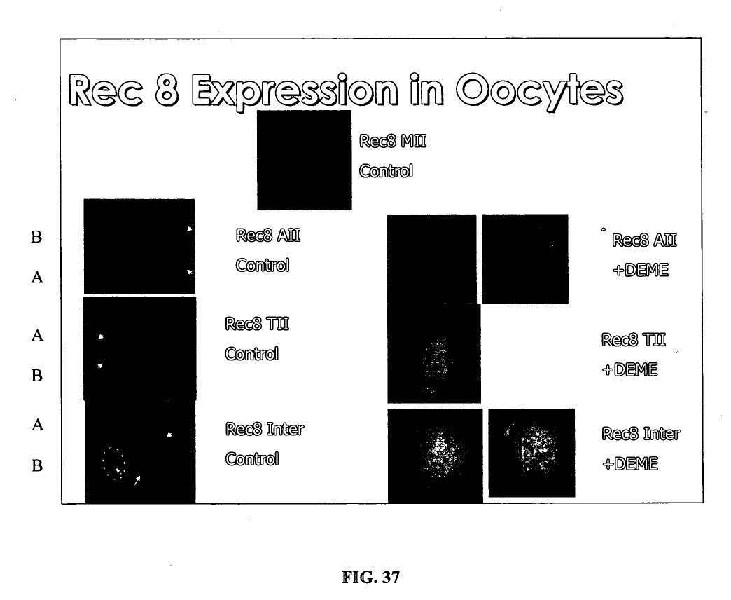

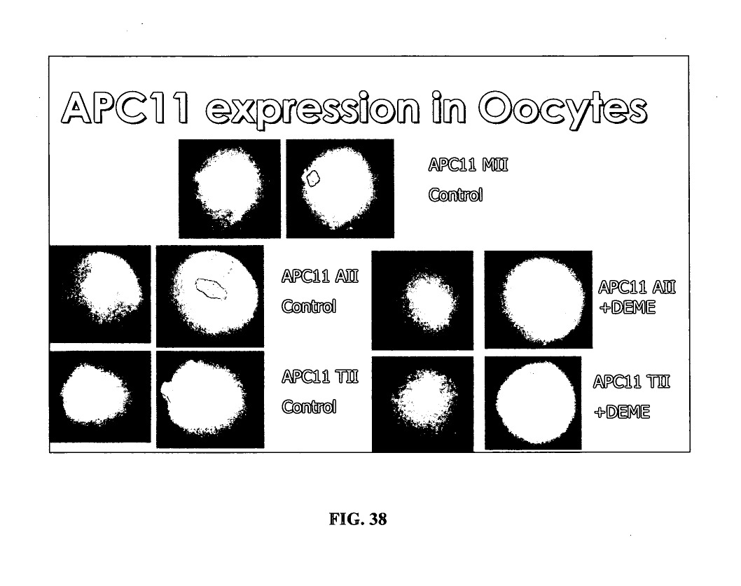

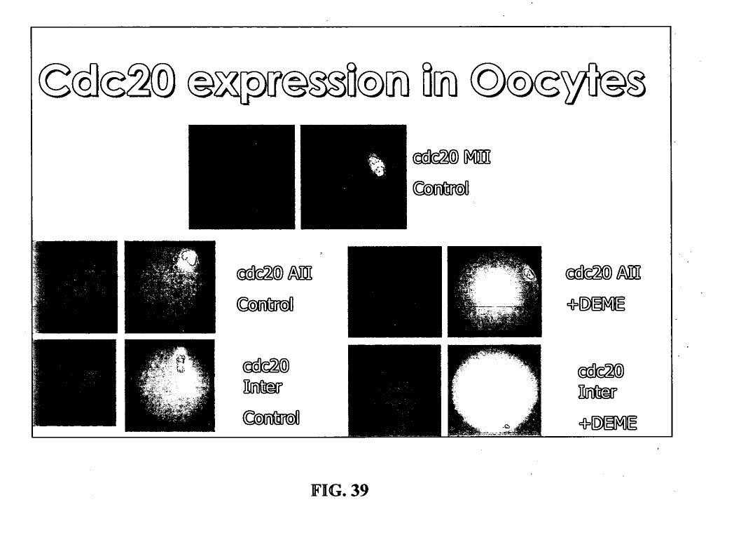

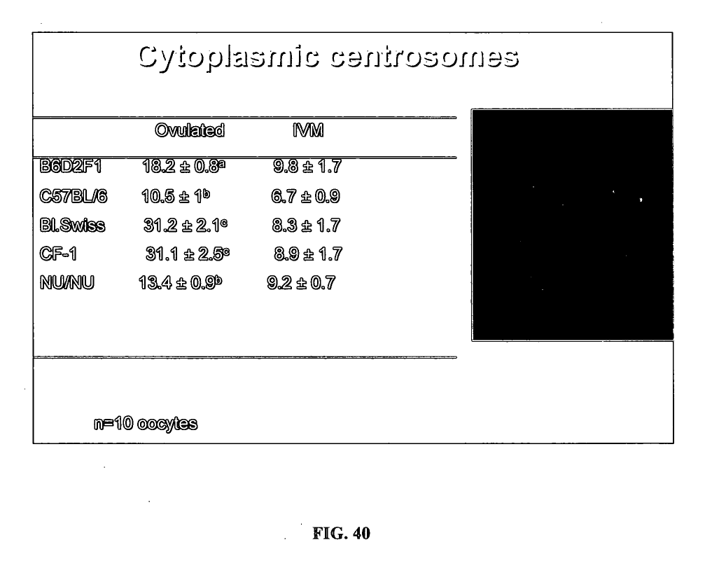

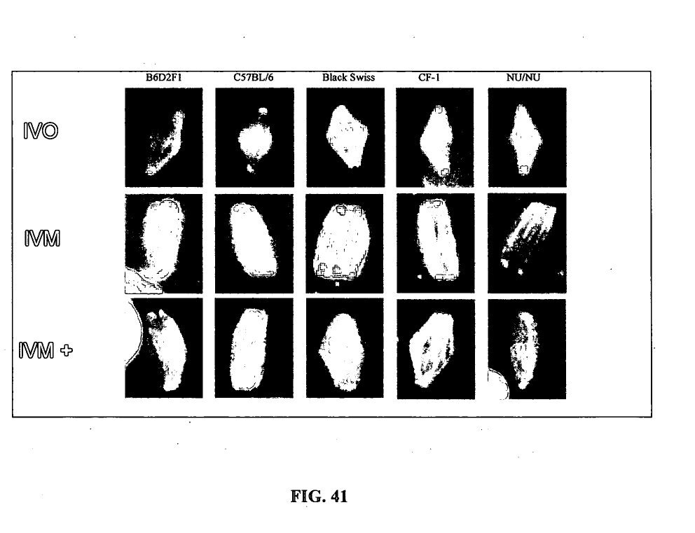

During Anaphase, when the Rec8 normally demonstrates cortical staining directly above the chromatin, Rec8 appears to aggregate in an area surrounding the chromatin, but not directly over it. Later in development the Rec8 staining pattern changes. Interestingly, during Telophase, Rec8 moves from an area near the chromatin to direct colocalization (purple spots) with the chromatin (FIG. 29; yellow arrows). This direct localization continues into Interphase as demecolcine induces the cell to extrude its chromatin in the second polar body (see FIG. 30).

### The Effects of Demecolcine on the Apc

Demecolcine has previously been used to assist in the enucleation of mammalian oocytes for nuclear transfer experiments. In order to better understand the efficacy of this process in early development, three key cell cycle regulation proteins (Apc11, Cdc20 and Rec8) were localized in developing mouse embryos in the presence or absence of demecolcine. It was the working hypothesis of this project that, since these three proteins have been previously described to associate with the meiotic spindle (Harper et al., 2002; Acquaviva et al., 2004; Castro et al., 2005), the disruption of the spindle would affect the localization of these proteins.

As described earlier, the data from the Apc11 localization experiments suggest striking effects of demecolcine on Apc localization. Although Apc11 strongly localized to the perispindular region of the cytoplasm in control oocytes (those incubated in the absence of demecolcine), in demecolcine-treated eggs, no localization was observed. From this data, one can postulate that without a well organized meiotic spindle, the Anaphase-promoting complex has nothing around which it will conglomerate. Thus, the localization of Apc11 may be tied directly to the integrity of the meiotic spindle.

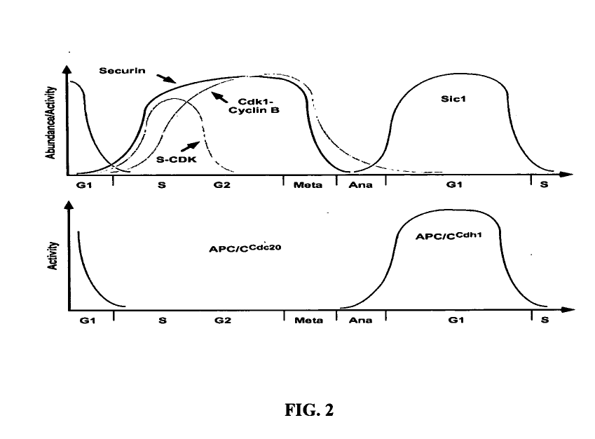

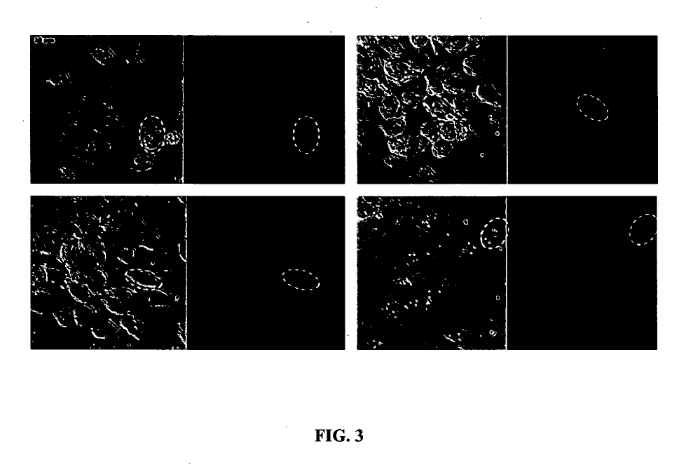

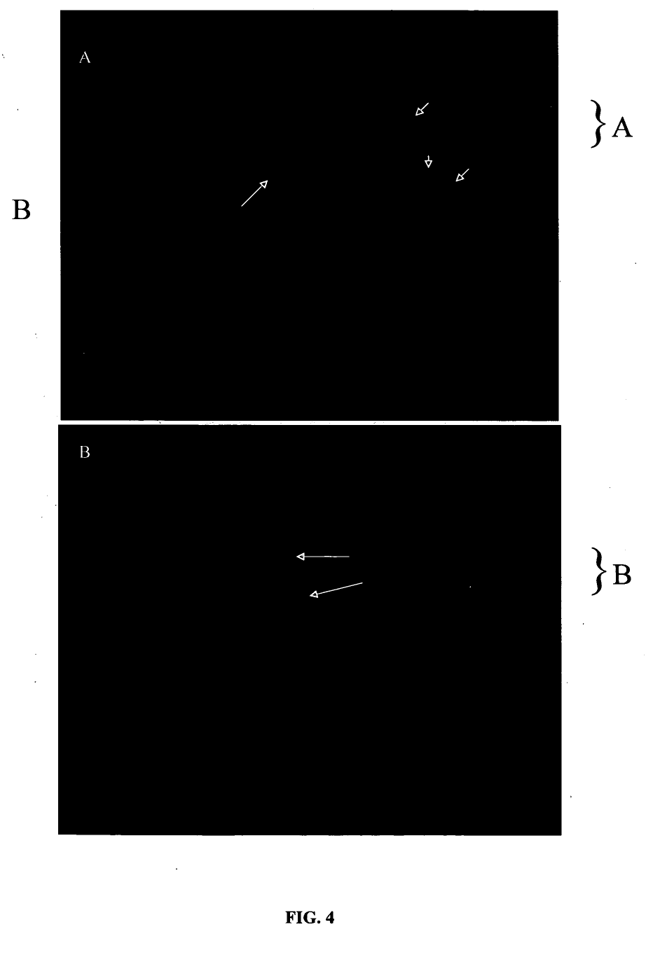

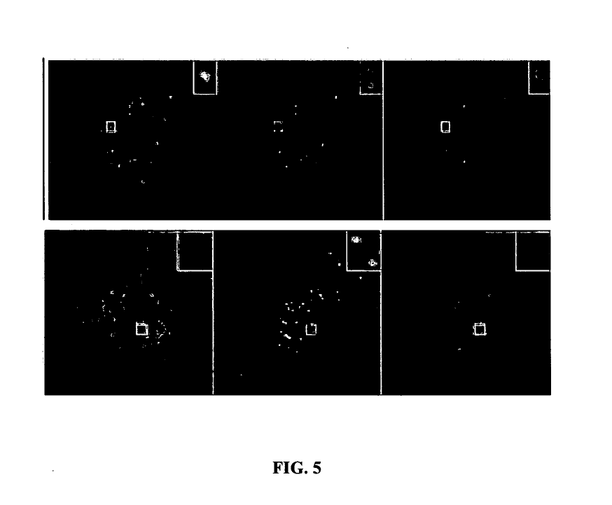

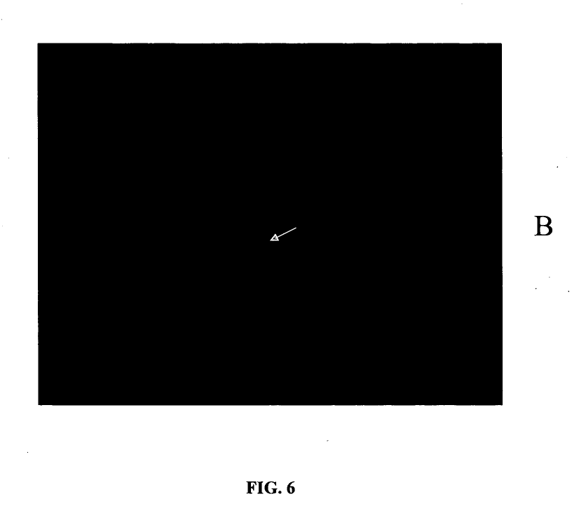

Although Cdc20 was not shown to localize to the meiotic spindle in control oocytes, the data from the Cdc20 localization experiments also show pronounced effects of demecolcine on the Apc. In the control activation, Cdc20 localization consistently weakened from punctate cytoplasmic staining at MII to a dim, diffuse pattern after activation. Cdc20 localization continued to diminish through TII only to intensify again at Interphase. This cyclic pattern is consistent with previous studies regarding Cdc20's role as both activator and substrate of the Anaphase-promoting complex (Zacharaie et al., 1999; and many others) (see FIG. 2.). However, in oocytes incubated in demecolcine, no developmental variation could be detected. Therefore, one could conjecture that demecolcine has in some way affected the Apc's ability to function properly in the orderly binding and destruction of Cdc20.

It is possible that a loss of Apc localization, caused by the disruption of the spindle, could be followed by a concomitant loss of Apc activity. It has been hypothesized that the Apc ubiquitinates proteins by bringing them into direct contact with the E2 enzyme (Harper, et al., 2002; Kraft et al., 2003; Passmore & Barford, 2004; Castro et al., 2005). In order to do this, it is necessary for the Apc to be in direct contact with the substrate. By reducing the concentration of Apc11 around segregation chromosomes (as seen in FIG. 14 through FIG. 16) an incubation in demecolcine could limit the ability of the complex to make contact with substrates in that area, thus, preventing ubiquitination of key cell cycle proteins. While this may or may not affect the ubiquitination of Securin, which has yet to be localized to the meiotic spindle, it could certainly affect cyclin B, whose destruction inactivates MPF, and other substrates vital to the regulation of the cell cycle known to localize to the perispindular region.

In order to test directly the effects of demecolcine on Apc activity, a series of ubiquitination and deubiquitination assays could be designed. Based on the protocol by Rape et al. (2006), the ability of the Apc to ubiquitinate Apc substrates could be closely examined both in the presence and absence of demecolcine. Given the effect of demecolcine on Apc localization, one would predict that the ubiquitination of several cell cycle proteins in the vicinity of the meiotic spindle would also be affected while the ubiquitination of those not localized to the spindle would not be severely disturbed. If the results of such experiments did in fact show a disruption or reduction in the ubiquitination of cyclin B (localized to the spindle), but not Securin (evenly distributed throughout the cytoplasm), it could provide further evidence that the Apc-regulated addition of ubiquitin is indeed a proximity reaction.

The data from the Rec8 localization experiments also indicate a subtle consequence of an incubation in demecolcine. In control oocytes, Rec8 consistently localized to the region of the cortex directly above chromosomal DNA. However, in demecolcine-treated oocytes, Rec8 appears to amass to the area directly surrounding the chromatin, not in the cortical region above it. Additionally, Rec8 localization appears in a direct colocalization with the chromatin following activation. Since this direct colocalization was not observed in control cells, one can postulate that perhaps the meiotic spindle in some way shields Rec8 from the DNA and the destruction of that spindle frees the chromatin from the protection of the spindle.

With regard to the use of Rec8 as an indirect indicator of Apc activity, these results could be interpreted in several ways. Although Rec8 did follow a consistent staining pattern in control eggs, this pattern differed slightly from the localization expected. The main function of Rec8 has been shown in cells to maintain the cohesion complex structure at the metaphase plate. The cleavage of Rec8 at the onset of Anaphase opens the cohesion ring, allowing the proper segregation of sister chromatids (reviewed by Revenkova & Jessberger, 2005). Given this function, one would predict a strong colocalization with cellular chromatin at Metaphase, which would disappear at the onset of Anaphase. Since this direct localization and destruction is not observed, it is possible that Rec8 might serve a different function in the mouse oocyte system and Rec8 would have been a less-than-ideal choice for an indirect indicator of Apc activity.

The differences between the predicted and observed Rec8 data are not all that surprising. The prediction was originally based on observations made of Scc1, another member of the kleisin family, in cells undergoing Mitosis, not Meiosis. At mitotic Metaphase, Scc1 localizes directly with the condensed chromatin. At the onset of mitotic Anaphase, Scc1 is cleaved by separase, the same enzyme known to be responsible for the excision of Rec8 in Meiosis, and disperses throughout the cytoplasm where is it degraded. Because of the similarities between Rec8 and Scc1, one would predict that the two proteins would localize in a similar manner. However, a different localization pattern was observed for Rec8. Perhaps this pattern can be attributed to the inherent differences in the meiotic versus mitotic mechanisms, among which are the symmetrical division of the cytoplasmic material, the idiosyncratic gene expression of meiotic cells, and dissolution of SMC complexes in oocytes from arm to centromere (Reviewed by Revenkova & Jessberger, 2005).

### Implications for SCNT

The data presented in the three localization experiments has implications for the field of somatic cell nuclear transfer (SCNT). Recently, demecolcine has been used to chemically assist the enucleation of oocytes for the purposes of mammalian cloning in several species. While this process has been shown to produce healthy cloned offspring slightly more efficiently than conventional enucleation methods, the overall efficiency of such procedure remains low. Since the majority of the data presented herein indicates that demecolcine could have a deleterious affect on the localization of the Apc, it is conceivable that this could contribute to the low efficiency by reducing the developmental competence of the donor oocytes. Therefore, it would be advantageous to develop a protocol that would continue to exploit the enucleation ability of demecolcine to assist oocytes in the extrusion of DNA while simultaneously maintaining a high functional activity of the Anaphase-promoting complex.

In order to test the effectiveness of such a protocol, ubiquitin assays similar to those described by Rape et al. (2006) can be completed on lysates of oocytes enucleated by conventional methods and assisted by demecolcine. If the Apc is shown to be more active in cells enucleated with demecolcine, this could help to explain the higher efficiency of chemical assisted enucleation.

### Example 2

### Aurora Kinases

### Protocol

1. Oocytes were collected from stimulated follicles 10 hp hCG injection at Telophase I stage.
2. Samples were arbitrarily divided into 3 groups.
   a. control KSOM⁺ media only.
   b. control KSOM⁺ media with 250 nM0 Hesperadin analog.
   c. experimental KSOM⁺ media with 250 nM Hersperadin.
3. Groups were incubated at 37° C., 5% CO₂ for 6 hours.
4. At 6 hours post-harvest (Metaphase II stage) half of each group was fixed in PFA, and the other half was washed and transferred to fresh KSOM-media with SrCl2 with appropriate treatment.

### Part I: Hesperadin Treatment

Hesperadin, an inhibitor of Aur kinases, has been used to investigate the effect of Aur kinase inhibition in cancer cells, but not in germ cells. The objective of this study was to assess the effect of Aur inhibition on mouse oocytes during Meiosis II and progression to Interphase. Oocytes were collected from preovulatory Graafian follicles of stimulated CF-1 mice at 10 h post-hCG. Cumulus denuded oocytes were either fixed, or cultured for 6 or 10 h+/−Hesperadin and fixed. Samples were processed for immunofluorescence microscopy using markers of spindle morphology (tubulin) and AurB kinase activity (pH3). Marked differences were observed in cultured oocytes treated with 250 nM Hesperadin after 6 h and 10 h. Although control oocytes (no treatment, or treatment with inactive Hesperadin analog) displayed a normal MII morphology, oocytes cultured for 6 h in 250 nM Hesperadin displayed disorganized spindles and scattered chromatids with complete lack of pH3 staining. Following SrCl₂ activation and further culture, control oocytes displayed normal phenotypic and temporal progression to Interphase. In contrast, oocytes cultured for 10 h in 250 nM Hesperadin revealed extensive disruption of the microtubule organization. There was no evidence of a spindle, but tubulin had arranged into a complex matrix throughout the cell, and extrusion of the second polar body (PB) had not occurred. As in control oocytes, the DNA was decondensed, and histone H3 was dephosphorylated. When compared to control oocytes, the pro-nucleus produced at Interphase was larger, presumably due to the lack of PB extrusion. These observations demonstrate the breadth of Aur kinase involvement in mechanisms that regulate meiotic progression and M phase exit in mouse oocytes. Mouse oocytes were collected at the Telophase I stage and in vitro matured in media with and without 250 nM concentration of Herperadin. Following maturation and activation, fixation was carried out using a 1% paraformaldehyde solution. Samples were prepared for immunofluorescence analysis using a pH3 polyclonal antibody, α/β tubulin monoclonal antibody and appropriate alexa fluor secondary antibodies.

### Results

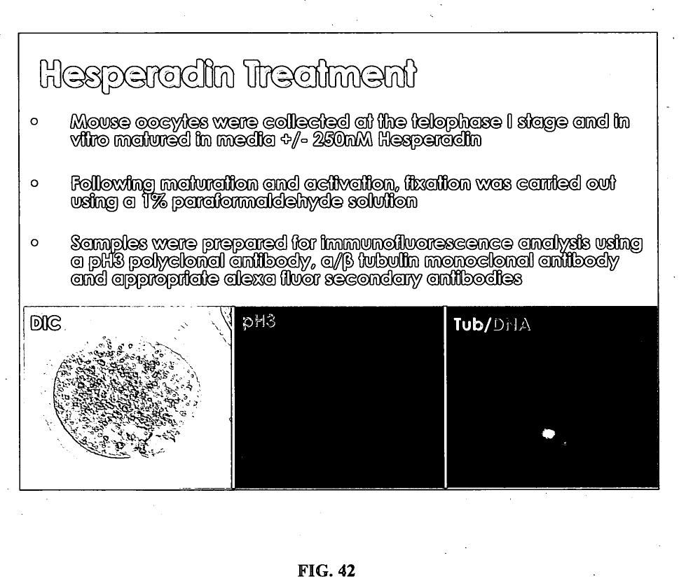

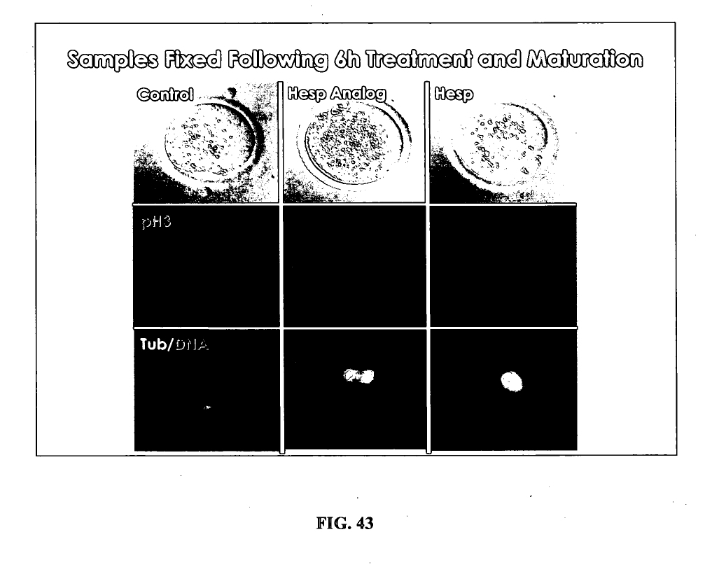

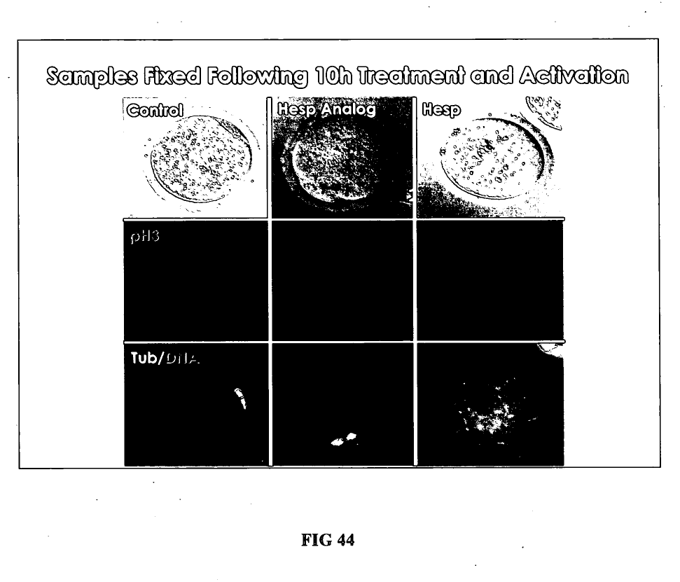

As is demonstrated in the FIGS. 42-44 Aurora B was sufficiently inhibited with Herperadin treatment. The observed phenotype showed spindle defects by Metaphase II, failure of karyokinesis with lack of 2^{nd} polar body formation and progression of the cell cycle. Using standard criteria, the oocytes appear morphologically normal and viable under DIC visualization. This experiment demonstrates that Aurora inhibition has a profound effect on the developmental competence of oocytes.

### Fixation of Oocytes and Processing for Immunofluorescence Analysis

At defined time-points after activation, control and demecolcine-treated oocytes can be fixed and extracted for 30 min at 37° C. in a buffer of 1% paraformaldehyde and 0.15% Triton X-100 in FHM media. Fixed oocytes can then be stored until processing at 4° C. in a PBS blocking solution containing 1% BSA, 0.2% powdered milk, 2% normal goat serum, 0.1 M glycine, 0.2% sodium azide and 0.01% Triton X-100. Wickramasinghe D, et al., Dev Biol 152: 62-74 (1992).

A multiple labeling protocol can be used for the detection of microtubules, microfilaments and chromatin by fluorescence microscopy as well as detection of spindle associated factors.

Labeled oocytes can then be examined using a Zeiss IM-35 inverted epi-fluorescence microscope fitted with filters selective for Hoechst, fluorescene and Texas Red and a 50 W mercury lamp. Selected images were acquired using a Photometrics Cool Snap CCD camera (Roper Scientific Inc., Trenton, N.J.) running on Metamorph software (version 5.0, Universal Imaging Corp., Downington, Pa.).

Part 2: mRNA Levels of Aurora A, B, C in Oocytes

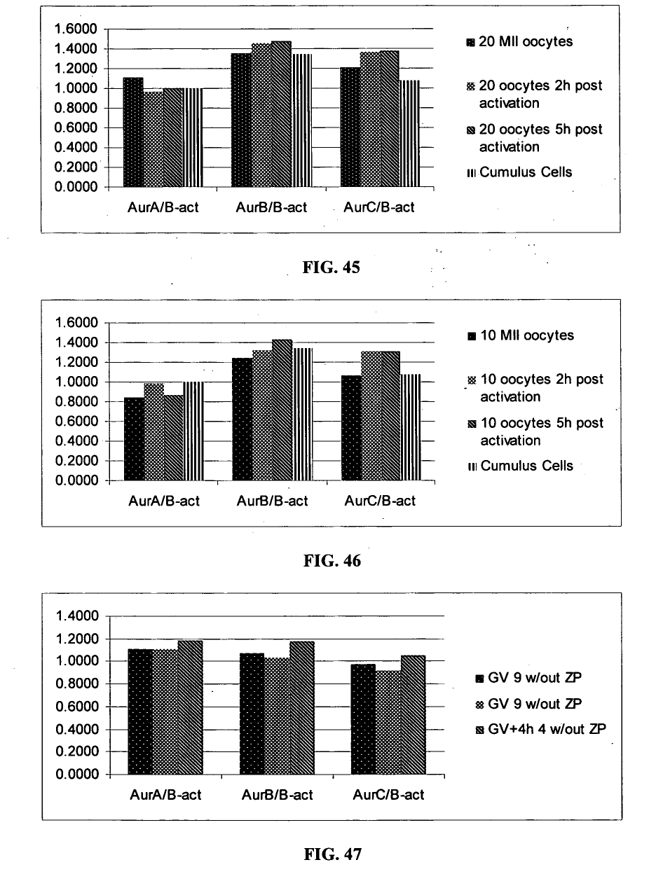

mRNA was extracted and quantified in oocytes using conventional methods including RT-PCR. Using specific primers for Aur A, B and C, levels of Aurora mRNAs were determined and compared with beta actin (B-act, served as a reference control, see FIGS. 45-47). Message was present for all auroras in GV, MII oocytes and increased in strontium chloride activated MII oocytes. Aur B and Aur C mRNA levels were most elevated particularly following activation. These data suggest that aurora kinases are present and very likely important enablers of cell cycle competence in oocytes. Thus, the functional presence of both Aur mRNAs and proteins associated with the oocyte spindle apparatus plays a key role in sustaining developmental competence of nuclear transfer embryo.

B-act F2xR2

| Sample | Mean | Std Dev | Median | Pixels |
|---|---|---|---|---|
| MII 20 w/ZP | 67.01 | 9.62 | 65 | 7920 |
| MII 10 w/ZP | 56.67 | 4.39 | 57 | 7920 |
| Cumulus Cells | 68.26 | 13.86 | 65 | 7920 |
| MII 10 w/out ZP | 63.65 | 5.22 | 63 | 7920 |
| MII 20 w/out ZP | 65.03 | 6.81 | 65 | 7920 |
| 2hpa 10 w/out ZP | 64.81 | 7.04 | 65 | 7920 |
| 2hpa 20 w/out ZP | 63.22 | 5.44 | 63 | 7920 |
| 5hpa 10 w/out ZP | 61.63 | 5.15 | 61 | 7920 |
| 5hpa 20 w/out ZP | 61.78 | 5.22 | 61 | 7920 |
| 5hpa 3 w/out ZP | 56.66 | 3.25 | 57 | 7920 |
| Water | 59.56 | 3.78 | 60 | 7920 |
| GV 9 w/out ZP | 76.82 | 12.81 | 75 | 1568 |
| GV 9 w/out ZP | 83.93 | 8.37 | 83 | 1568 |
| GV + 4 h 4 w/out ZP | 79.78 | 14.66 | 79 | 1568 |

AurA F2xR2

| Sample | Mean | Std Dev | Median | Pixels |
|---|---|---|---|---|
| MII 20 w/ZP | 62.08 | 16.15 | 59 | 7920 |
| MII 10 w/ZP | 50.36 | 8.98 | 49 | 7920 |
| Cumulus Cells | 68.51 | 22.47 | 65 | 7920 |
| MII 10 w/out ZP | 53.93 | 8.43 | 52 | 7920 |
| MII 20 w/out ZP | 71.95 | 18.27 | 69 | 7920 |
| 2hpa 10 w/out ZP | 63.94 | 11.23 | 62 | 7920 |
| 2hpa 20 w/out ZP | 61.44 | 12.50 | 59 | 7920 |
| 5hpa 10 w/out ZP | 53.38 | 7.87 | 52 | 7920 |
| 5hpa 20 w/out ZP | 61.90 | 14.25 | 59 | 7920 |
| 5hpa 3 w/out ZP | 54.64 | 7.00 | 53 | 7920 |
| Water | 51.60 | 3.93 | 51 | 7920 |
| GV 9 w/out ZP | 85.09 | 19.85 | 81 | 1568 |
| GV 9 w/out ZP | 93.38 | 18.61 | 91 | 1568 |
| GV + 4 h 4 w/out ZP | 94.95 | 25.06 | 90 | 1568 |

AurB F3xR3

| Sample | Mean | Std Dev | Median | Pixels |
|---|---|---|---|---|
| MII 20 w/ZP | 81.05 | 9.29 | 80 | 7920 |
| MII 10 w/ZP | 70.75 | 4.47 | 71 | 7920 |
| Cumulus Cells | 91.64 | 15.15 | 88 | 7920 |
| MII 10 w/out ZP | 79.06 | 4.87 | 79 | 7920 |
| MII 20 w/out ZP | 87.96 | 10.62 | 86 | 7920 |
| 2hpa 10 w/out ZP | 85.50 | 8.61 | 85 | 7920 |
| 2hpa 20 w/out ZP | 91.67 | 10.15 | 90 | 7920 |
| 5hpa 10 w/out ZP | 88.27 | 7.24 | 88 | 7920 |
| 5hpa 20 w/out ZP | 91.49 | 10.95 | 89 | 7920 |
| 5hpa 3 w/out ZP | 80.67 | 5.30 | 80 | 7920 |
| Water | 83.57 | 4.36 | 83 | 7920 |
| GV 9 w/out ZP | 82.30 | 18.12 | 78 | 1568 |
| GV 9 w/out ZP | 87.00 | 18.30 | 84 | 1568 |
| GV + 4 h 4 w/out ZP | 93.91 | 26.12 | 89 | 1568 |

AurC FxR

| Sample | Mean | Std Dev | Median | Pixels |
|---|---|---|---|---|
| MII 20 w/ZP | 67.45 | 11.62 | 66 | 7920 |
| MII 10 w/ZP | 63.15 | 7.29 | 63 | 7920 |
| Cumulus Cells | 73.70 | 11.86 | 73 | 7920 |
| MII 10 w/out ZP | 67.72 | 6.13 | 67 | 7920 |
| MII 20 w/out ZP | 79.06 | 9.88 | 78 | 7920 |
| 2hpa 10 w/out ZP | 84.95 | 11.44 | 84 | 7920 |
| 2hpa 20 w/out ZP | 86.69 | 10.07 | 86 | 7920 |
| 5hpa 10 w/out ZP | 80.84 | 7.49 | 80 | 7920 |
| 5hpa 20 w/out ZP | 85.30 | 10.23 | 84 | 7920 |
| 5hpa 3 w/out ZP | 72.89 | 3.71 | 73 | 7920 |
| Water | 74.81 | 3.72 | 75 | 7920 |
| GV 9 w/out ZP | 75.27 | 7.44 | 74 | 1568 |
| GV 9 w/out ZP | 76.9 | 5.69 | 77 | 1568 |
| GV + 4 h 4 w/out ZP | 83.82 | 15.87 | 83 | 1568 |

AurC FxR2

| Sample | Mean | Std Dev | Median | Pixels |
|---|---|---|---|---|
| MII 20 w/ZP | 42.38 | 5.06 | 42 | 7920 |
| MII 10 w/ZP | 48.87 | 12.34 | 47 | 7920 |
| Cumulus Cells | 36.13 | 2.92 | 36 | 7920 |
| MII 10 w/out ZP | 46.56 | 8.00 | 46 | 7920 |
| MII 20 w/out ZP | 57.82 | 16.10 | 56 | 7920 |
| 2hpa 10 w/out ZP | 55.14 | 10.74 | 54 | 7920 |
| 2hpa 20 w/out ZP | 56.88 | 12.12 | 56 | 7920 |
| 5hpa 10 w/out ZP | 44.59 | 3.26 | 45 | 7920 |
| 5hpa 20 w/out ZP | 66.44 | 21.53 | 61 | 7920 |
| 5hpa 3 w/out ZP | 47.98 | 4.26 | 47 | 7920 |
| Water | 72.24 | 9.98 | 73 | 7920 |
| GV 9 w/out ZP | 85.16 | 26.16 | 79 | 1568 |
| GV 9 w/out ZP | 75.73 | 17.06 | 72 | 1568 |
| GV + 4 h 4 w/out ZP | 66.83 | 21.25 | 59 | 1568 |
| MM 10 w/ZP | 0.8887 | 1.2485 | 1.1143 | 0.8624 |
| Sample | AurA/B-act | AurB/B-act | AurC/B-act | AurC/B-act |
| 10 MII oocytes | 0.8473 | 1.2421 | 1.0639 | 0.7315 |
| 10 oocytes 2 h post | 0.9866 | 1.3192 | 1.3108 | 0.8508 |
| 10 oocytes 5 h post | 0.8661 | 1.4323 | 1.3117 | 0.7235 |
| Cumulus Cells | 1.0037 | 1.3425 | 1.0797 | 0.5293 |
| MII 20 w/ZP | 0.9264 | 1.2095 | 1.0066 | 0.6324 |
| 20 MII oocytes | 1.1064 | 1.3526 | 1.2157 | 0.8891 |
| 20 oocytes 2 h post | 0.9718 | 1.4500 | 1.3712 | 0.8997 |
| 20 oocytes 5 h post | 1.0019 | 1.4809 | 1.3807 | 1.0754 |
| Cumulus Cells | 1.0037 | 1.3425 | 1.0797 | 0.5293 |
| GV 9 w/out ZP | 1.1077 | 1.0713 | 0.9798 | 1.1086 |
| GV 9 w/out ZP | 1.1126 | 1.0366 | 0.9162 | 0.9023 |
| GV + 4 h 4 w/out ZP | 1.1901 | 1.18 | 1.05063926 | 0.8377 |
| 5hpa 3 w/out ZP | 0.9643 | 1.4238 | 1.2864 | 0.8468 |
| Water | 0.8664 | 1.4031 | 1.2560 | 1.2129 |

Additional References: Wakayama & Yanagimachi, Mol Reprod Dev. April; 58(4):376-83 (2001) Eggan et al., Proc Natl Acad Sci USA. May 22; 98(11):6209-14. 2001; Rosa et al., MBC, March; 17(3):1483-93 (2006) All references disclosed herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of forming a nuclear transfer embryo, comprising:
a) obtaining an enucleated oocyte;
b) containing and maintaining an effective amount of spindle associated factors in the enucleated oocyte; and
c) combining the enucleated oocyte and at least the nucleus of a donor cell of the same species of said oocyte, thereby forming a nuclear transfer embryo.

2. The method of claim 1, wherein the spindle associated factors are selected from the group consisting of: Aurora kinase A, Aurora kinase B, Aurora kinase C, Survivin, Securin, INCEP, Borealin/Dasra B, gamma tubulin, pericentrin, members of the Rec8 family proteins, Cdc20, members of the Anaphase Promoting Complex (Apc), the Polo kinases, Feo/Klp3A, Apc11, cohesin, MEI-S322, spindle checkpoint proteins, Bub1, Bub3, BubR1, Mad1, Mad2 and CENP-E and combinations thereof.

3. The method of claim 1, wherein the oocytes is enucleated with a chemical selected from the group consisting of demecolcine, paclitaxel, phalloidin, colchicine, and nocodozole.

4. The method of claim 3, further includes activating the oocyte prior to exposing the oocyte to said chemical.

5. The method of claim 1, wherein the oocyte is mammalian.

6. The method of claim 5, wherein the mammal is non-human.

7. A method of cloning a mammal, comprising:
a) obtaining an enucleated oocyte;
b) maintaining an effective amount of spindle associated factors in the enucleated oocyte;
c) combining the oocyte with at least the nucleus of a donor cell of the same species of said oocyte prior to cessation of extrusion of the second polar body from said oocyte, thereby forming a nuclear transfer embryo;
d) impregnating a mammal of the same species as the nuclear transfer embryo with the nuclear transfer embryo under conditions suitable for gestation of the cloned mammal; and
e) gestating the embryo, thereby causing the embryo to develop into the cloned mammal.

8. The method of claim 7, wherein the oocyte is enucleated with a chemical selected from the group consisting of demecolcine, paclitaxel, phalloidin, colchicine, and nocodozole.

9. A method of producing a transgenic mammal, comprising:
a) destabilizing microtubules of an oocyte, whereby essentially all endogenous genetic material collects at a second polar body during Meiosis of said oocyte and maintaining spindle associated factors in resulting enucleated oocyte;
b) combining the oocyte with at least the nucleus of a donor cell of the same species of said oocyte prior to cessation of extrusion of the second polar body for said oocyte, thereby forming a nuclear transfer embryo;
c) impregnating a mammal of the same species as the nuclear transfer embryo with the nuclear transfer embryo under conditions suitable for gestation of the transgenic mammal; and
d) gestating the embryo, thereby causing the embryo to develop into the transgenic mammal.

10. The method of claim 9, wherein the oocyte is enucleated with a chemical selected from the group consisting of demecolcine, paclitaxel, phalloidin, colchicine, and nocodozole.

11. A method of producing a protein of interest in an animal, comprising,
a) destabilizing microtubules of an oocyte, whereby essentially all endogenous genetic material collects at a second polar body during Meiosis of said oocyte;
b) maintaining spindle associated factors in resulting enucleated oocyte;
c) combining the oocyte with at least the nucleus of a donor cell of the same species of said oocyte prior to cessation of extrusion of the second polar body from said oocyte, thereby forming a nuclear transfer embryo;
d) impregnating a mammal of the same species as the nuclear transfer embryo with the nuclear transfer embryo under conditions suitable for gestation of the cloned mammal;
e) gestating the embryo, thereby causing the embryo to develop into the cloned mammal; and
f) purifying the protein of interest from the cloned animal.

12. The method of claim 11, wherein the oocyte is enucleated with a chemical selected from the group consisting of demecolcine, paclitaxel, phalloidin, colchicine, and nocodozole.

13. A method of preparing an enucleated oocyte for nuclear transfer comprising:
a) obtaining an oocyte
b) enucleating the oocyte
c) maintaining an effective amount encleated oocyte; wherein the oocyte has efficiency increased competency and subsequent reconstruction with a donor nuclei.

14. The method of claim 13, wherein the spindle associated factors are selected from the group consisting of: Aurora kinase A, Aurora kinase B, Aurora kinase C, Survivin, Securin, INCEP, Borealin/Dasra B, gamma tubulin, pericentrin, members of the Rec8 family proteins, Cdc20, members of the Anaphase Promoting Complex (Apc), the Polo kinases, Feo/Klp3A, Apc11, cohesin, MEI-S322, spindle checkpoint proteins, Bub1, Bub3, BubR1, Mad 1, Mad2 and CENP-E and combinations thereof.
